(19) United States
(12) Patent Application Publication
(10) Pub. No.: US 2005/0019326 A1
(43) Pub. Date: Jan. 27, 2005

(54) C1q complement inhibitors and methods of use thereof

(75) Inventor: Gregory L. Stahl, Clinton, MA (US)

Correspondence Address: Helen C. Lockhart;Wolf, Greenfield & Sacks, P.C., 600 Atlantic Avenue, Boston, MA, 02210, US

(73) Assignee: The Brigham and Women's Hospital, Inc., Boston, MA (US)

(21) Appl. No.: 10/869,823

(22) Filed: Jun. 16, 2004

Related U.S. Application Data
(60) Provisional application No. 60/479,025, filed on Jun. 16, 2003.

(51) Int. Cl. A61K 39/395; C12N 5/06

(52) U.S. Cl. 424/145.1; 530/388.25; 435/326

(57) ABSTRACT

The present invention relates to agents that bind and/or inhibit classical complement C1 subcomponent, C1q, as well as methods of their use. In particular, the invention relates to methods for inhibiting classical complement activation by contacting a C1q molecule with a C1q inhibitor. The invention, therefore, also relates to methods for treating disorders using the C1q inhibitors described herein. More specifically, the C1q inhibitors provided can be used to treat any condition mediated by the classical complement system. More specifically, the C1q inhibitors can be used to treat inflammatory conditions and the resulting tissue injury, autoimmune disorders and cancer.

### RELATED APPLICATIONS

This application claims priority under 35 U.S.C. §119 from U.S. provisional application Ser. No. 60/479,025 filed Jun. 16, 2003, the entire contents of which is incorporated by reference.

### GOVERNMENT SUPPORT

Aspects of the invention may have been made using funding from the National Institutes of Health Grant number HL56086 and HL52886. Accordingly, the Government may have rights in the invention.

### FIELD OF THE INVENTION

The present invention relates to agents that bind and/or inhibit classical complement C1 subcomponent, C1q, as well as methods of their use. In particular, the invention relates to methods for inhibiting classical complement activation by contacting a C1q molecule with a C1q inhibitor. The invention, therefore, also relates to methods for treating disorders using the C1q inhibitors described herein.

### BACKGROUND OF THE INVENTION

The immune system functions to defend the body against pathogenic bacteria, viruses and parasites. Immunity against foreign pathogens usually involves the complement system. The complement system is a cascade of about 18 sequentially activated serum proteins which function to recruit and activate other cells of the immune system, effect cytolysis of target cells and induce opsonization of foreign pathogens. Complement can be activated by the presence of either antibody/antigen complexes, as in the classical complement pathway (CCP), or microbial surfaces, as in the alternative complement pathway or lectin pathway. In the CCP, antibody-antigen complexes activate the CCP and are bound by classical complement C1 subcomponent, C1q (FIG. 1). This in turn sets off a cascade of reactions involving CCP components C1r, C1s, C4 and C2. The reactions of the cascade lead to the enzymatic step of serum protein C3 cleavage to C3b and C3a which is a step common between the various complement pathways. This, in turn, initiates the terminal steps of complement function including the cleavage of C5 to C5b and C5a and subsequent deposition of C5b-C9 onto the target cell membrane.

### SUMMARY OF THE INVENTION

The invention relates to agents that bind and/or inhibit classical complement C1 subcomponent, C1q, as well as methods of their use. The agents that bind and/or inhibit classical complement C1q are agents such as peptides, polysaccharides, nucleosides, drugs, nucleoproteins, lipoproteins, carbohydrates, glycoproteins, steroids, lipids, and other biologically active materials. These agents also include small molecules. In some embodiments these agents do not inhibit classical complement activation. In other embodiments these agents do inhibit classical complement activation.

The agents provided herein are intended to include antibodies as well as antigen-binding fragments thereof. In one aspect of the invention a monoclonal antibody produced by a hybridoma cell line deposited under ATCC Accession No. PTA-5226 is provided. In another aspect of the invention the hybridoma cell line that produces the antibody is provided.

Other aspects of the invention provide antigen-binding fragments of the deposited antibody. These antigen-binding fragments can be a F(ab′)₂ fragment, a Fd fragment, a Fab fragment, or a Fv fragment. In yet another embodiment the antigen-binding fragment is a CDR. The CDR in some embodiments is a CDR1, CDR2 or CDR3. In other embodiments the antigen-binding fragment is a light chain CDR2 or a light chain CDR1. In still other embodiments the antigen-binding fragment is a heavy chain CDR2 or a heavy chain CDR1. In still further embodiments the antigen-binding fragment is a heavy chain or light chain CDR3. The antibodies and antigen-binding fragments provided herein are encompassed by the term “C1q binding peptides”. C1q binding peptides can include any combination of the light chain and/or heavy chain CDRs. The C1q binding peptides can include, in some embodiments, other amino acid residues that do not eliminate the ability of the C1q binding peptide to bind C1q. In other embodiments the additional amino acid residues do not eliminate the ability of the C1q binding peptide to bind C1q and inhibit classical complement activation. Therefore, in one embodiment fusion proteins are provided. In other embodiments 1, 2, 3, 4, 5, 6, 7, 8, 9, 10, 15, 20, 25, 30, 35, 40, 50, 60, 70, 80, 90, 100, 150, 200, 250, 300, 400, 500 or more amino acid residues are added to either end of the C1q binding peptide or both.

In another aspect of the invention C1q binding peptides are peptides which include a C1q binding region, which selectively binds to a human C1q epitope. A C1q binding region is any portion of a monoclonal antibody or a functional variant thereof, which specifically binds a human C1q epitope. Therefore, in one aspect of the invention a C1q binding peptide which selectively binds a human C1q epitope defined by the monoclonal antibody produced by the hybridoma cell line deposited under ATCC Accession No: PTA-5226 is provided.

C1q binding peptides, therefore, include peptides which comprise an antibody or antigen-binding fragment described above. C1q binding peptides, in some embodiments, comprise a F(ab′)₂ fragment, a Fd fragment, a Fab fragment, or a Fv fragment. In other embodiments the C1q binding peptides comprise a CDR1, CDR2 or CDR3 or a combination thereof. In some embodiments the isolated C1q binding peptide of the invention is an intact soluble monoclonal antibody. In still other embodiments the isolated C1q binding peptide is a humanized monoclonal antibody. In some embodiments the C1q binding peptides are isolated C1q binding peptides.

Functional variants of the C1q binding peptides described herein are also provided in some aspects of the invention. A “functional variant” as used herein is a peptide having the sequence of an antibody or antigen-binding fragment region with conservative substitutions therein.

In some aspects the C1q binding peptide binds to C1q and inhibits classical complement activation. Therefore, in these aspects a C1q binding peptide that is also considered a C1q inhibitor is provided.

In other embodiments the C1q binding peptide is bound to a therapeutic moiety. In further embodiments the therapeutic moiety is a drug. In yet other embodiments the drug is an anti-cancer agent, an anti-inflammatory agent, an immunosuppressant, an agent for treating an autoimmune disorder or another complement inhibitor. In still other embodiments the anti-cancer agent is an anti-cancer agent that induces apoptosis.

In other aspects of the invention a composition of any of the C1q binding peptides described herein is provided. In some embodiments the composition can further include a pharmaceutically acceptable carrier. In other aspects the composition includes a C1q inhibitor. The compositions provided in some embodiments further comprise a second agent (e.g., a drug) for the treatment of a classical complement mediated disorder. These drugs include other complement inhibitors. In some embodiments the classical complement mediated disorder is an inflammatory condition and the drug is an anti-inflammatory agent or an immunomodulator (e.g., immunosuppressant). In still other embodiments, the classical complement mediated disorder is cancer and the drug is an anti-cancer agent. In yet other embodiments the anti-cancer agent is one that induces apoptosis.

In other aspects of the invention clinical and therapeutic applications are provided. In one aspect a method for inhibiting classical complement activation, by contacting a C1q molecule with an effective amount of a C1q inhibitor or a composition thereof to inhibit classical complement activation is provided. In these aspects the C1q inhibitor or composition thereof is administered to a subject in an amount effective to inhibit classical complement activation. In some embodiments the subject is a mammal. In still other embodiments the subject is a human. Other subjects provided herein include non-human primates, cows, horses, pigs, sheep, goats, dogs, cats or rodents.

The invention, therefore, also relates to methods for treating disorders associated with classical complement activation using the C1q inhibitors or compositions thereof described herein through the inhibition of classical complement activation. In one aspect the disorder associated with classical complement activation is an inflammatory condition, and a subject with the inflammatory condition or at risk thereof is administered a C1q inhibitor or composition thereof in an amount effective to treat or prevent the inflammatory condition. In another aspect the disorder is an allergic condition. In another aspect the disorder is an autoimmune disorder and the subject has or is at risk of having the autoimmune disorder. In some embodiments, the autoimmune disorder is not arthritis (e.g., rheumatoid arthritis, collagen-induced arthritis), glomerulo-nephritis, lupus (e.g., system lupus erythematosus (SLE)), membranous nephropathy, or myasthenia gravis. In some embodiments the autoimmune disorder is not any of these diseases individually or in combination. In still another aspect a method of treating cancer by administering to a subject with cancer or at risk thereof a C1q inhibitor or a composition thereof and an anti-cancer agent that induces apoptosis in an amount effective to treat the cancer is provided. The cancer can be any cancer (e.g., bladder cancer, pancreatic cancer, lung cancer, kidney cancer, sarcoma, breast cancer, brain cancer, colon cancer, liver cancer, stomach cancer, oral cancer, ovarian cancer, prostate cancer, testicular cancer, or melanoma). In still other aspects a method of treating ischemia reperfusion is provided. Ischemia reperfusion can occur in many organs, such as the brain, lungs and kidneys. Ischemia reperfusion can also occur in skeletal muscle. Therefore, methods of treating ischemia reperfusion in any of these organs are provided herein.

The effectiveness of a therapeutic agent can be reduced and/or treatment with a therapeutic agent can result in undesired side effects because of inflammation resulting from the administration of the therapeutic agent. The reduction in efficacy and/or the occurrence of unwanted side effects can occur from the body's reaction to the therapeutic agent, to a delivery agent, such as a liposome or other lipid formulation that contains the therapeutic agent, or both. The undesired side effects include allergic reactions. Therefore, in another aspect of the invention the C1q inhibitors or compositions thereof described herein are used to decrease inflammation to improve the effectiveness and/or desirability of therapy with a therapeutic agent. In this aspect of the invention, C1q inhibitors are administered along with the therapeutic agent and/or delivery agent that results in inflammation and/or unwanted side effects. The therapeutic agent can be any therapeutic agent that results in inflammation and/or the side effects associated with inflammation. The delivery agent can be any delivery agent that causes the unwanted effects. In some embodiments the delivery agent is a liposome or other lipid formulation and the method includes administering a C1q inhibitor with the delivery of any therapeutic agent in a liposome. In some embodiments the therapeutic agent is taxol, a radio-opaic agent, an oligonucleotide, a chimeric antibody, a xenograft, etc. The C1q inhibitor can be administered at the same time, prior to or subsequent to the administration of the therapeutic agent.

In the above methods the C1q inhibitor can be a C1q binding peptide but is not necessarily so. In certain embodiments the C1q binding peptide is derived from the deposited monoclonal antibody provided herein. In other embodiments the C1q inhibitor is C1-Inhibitor (C1-INH).

In some embodiments, the C1q binding peptides provided are encoded by nucleic acid molecules that are 90%, 95%, 97% or 99% homologous to the nucleic acid that encodes the deposited monoclonal antibody or a fragment thereof. In other embodiments, the amino acid sequence of the C1q binding peptides provided shares at least 70%, 75%, 80%, 85%, 90%, 95%, 97%, or 99% identity with the amino acid sequence of the deposited monoclonal antibody or a fragment thereof. In still other embodiments, the C1q binding peptides provided include peptides that comprise the amino acid sequence of the deposited monoclonal antibody or a fragment thereof with conservative substitutions therein. The C1q binding peptides derived from the sequence of the deposited monoclonal antibody or a fragment thereof can contain 1, 2, 3, 4, 5, 6, 7, 8, 9, 10, 11, 12, 13, 14, 15, 16, 17, 18, 19, 20, 21, 22, 23, 24, 25, 30, 40, 50 or more substitutions.

In the various methods of treatment described herein the C1q inhibitor can be administered with another therapeutic agent. The C1q inhibitor and other therapeutic agent can be administered concomitantly in some embodiments. In other embodiments the C1q inhibitor is administered before or after the other therapeutic agent.

In still another aspect of the invention a method for detecting the presence of C1q in a sample is provided. The method includes the steps of contacting the sample with an isolated C1q binding peptide provided herein and detecting the isolated C1q binding peptide-C1q complex, wherein the presence of a complex in the sample is indicative of the presence of C1q in the sample.

Each of the limitations of the invention can encompass various embodiments of the invention. It is, therefore, anticipated that each of the limitations of the invention involving any one element or combinations of elements can be included in each aspect of the invention.

### BRIEF DESCRIPTION OF THE DRAWINGS

This application includes drawings which illustrate various aspects of the invention; however, the drawings are not required for enablement of the claimed invention.

FIG. 1 is a schematic depicting the antigen/antibody-dependent classical complement pathway as well as the antibody-independent alternative and lectin complement pathways. All three pathways merge at C3 and lead to the formation of the terminal complement complex (C5b-9).

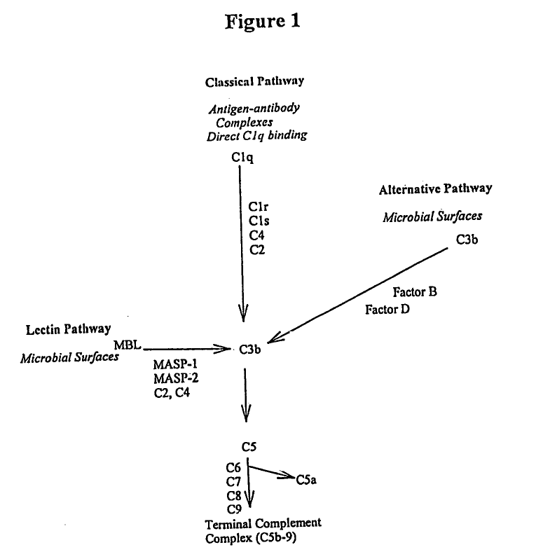

FIG. 2 provides the results of the anti-human C1q hemolytic complement assay (CH₅₀).

FIG. 3 provides the results of the MBL-dependent C3 deposition enzyme-linked immunosorbent assay (ELISA).

FIG. 4 provides the results of the immunoprecipitation (IP) of human C1q (huC1q) from sera using anti-huC1q (P1 H10). Lane 1—IP of huC1q, NR. Lane 2—IP of huC1q isolated from human sera, NR. Lane 3—BioRad Broad Range SDS-Page pre-stained molecular weight marker. Lane 4—IP of huC1q, R. Lane 5—IP of huC1q isolated from human sera, R.

FIG. 5 provides the representative sensogram of the binding of anti-huC1q P1 H10 to immobilized huC1q.

### DETAILED DESCRIPTION OF THE INVENTION

The invention relates to agents that bind and/or inhibit classical complement C1 subcomponent, C1q, as well as methods of their use. These methods include methods for inhibiting classical complement activation by contacting a C1q molecule with a C1q inhibitor. The invention, therefore, also relates to methods for treating disorders associated with classical complement activation using the C1q inhibitors described herein through the inhibition of classical complement activation.

The invention is based, in part, on the discovery of a molecule that functionally inhibits the classical complement pathway by interfering with C1 activation by binding to human complement C1q. The C1q inhibitor described herein can be used in a variety of therapeutic and clinical applications. The C1q inhibitors, provided herein, can be used for any classical complement-associated disorder, wherein the inhibition of the classical complement pathway is desired. For example, in certain inflammatory conditions including, but not limited to, autoimmune diseases, the classical pathway is pro-inflammatory, and the inhibition of C1q would decrease inflammation and tissue injury.

The invention is also based, in part, on the discovery that the classical complement pathway is responsible for removal of apoptotic cells in the body by a non-inflammatory mechanism; therefore, the C1q inhibitors can be used to inhibit the classical complement pathway but allow the mannose binding lectin (MBL) pathway to remove apoptotic cells. As the MBL pathway initiates an inflammatory/destructive cell removal process, this would lead to an exaggerated anti-tumor response. Anti-C1q therapy can, therefore, be used as a cancer therapy or as an adjunct to cancer therapy, where one would induce apoptosis of the cancer cells and inhibit C1q, thereby initiating MBL pathway activation, resulting in killing of the apoptotic cells and neighboring cancer cells.

The invention, therefore, provides, in part, C1q inhibitors. “C1q inhibitors” are molecules which bind to human C1q and inhibit classical complement activation. The C1q inhibitors may function by blocking the binding of C1q to an antibody-antigen complex or by blocking the deposition of other classical complement components associated with C1q, or in other words, blocking the formation of the C1 complex (the association of C1q with C1r and C1s). C1q inhibitors also include molecules which bind to the C1q complex and inhibits its activation. In one embodiment, the C1q inhibitor is C1-Inhibitor (C1-INH). C1q inhibitors also include molecules that bind C1q in a way that inhibits C1-INH from coming off of the C1 complex. C1q inhibitors, therefore, may function by preventing interactions of C1q with any molecule or complex of molecules, wherein the interactions can lead to classical complement pathway (CCP) activation in the absence of the C1q inhibitor. A molecule or complex of molecules which interact with C1q is also referred to herein as a “C1q binding ligand”.

C1q inhibitors include, b but are not limited to, peptides, polysaccharides, nucleosides, drugs, nucleoproteins, lipoproteins, carbohydrates, glycoproteins, steroids, lipids, and other biologically active materials. C1q inhibitors, therefore, are also intended to include small molecules. “C1q binding peptides” are peptides that include a C1q binding region which specifically binds to human C1q. These peptides include peptides that inhibit classical complement activation as well as peptides that do not inhibit classical complement activation. Included in this definition are the C1q binding peptides provided herein as well as those with conservative substitutions therein. C1q binding peptides also include peptides that further include a label, a therapeutic agent, additional amino acid residues, or any molecule that does not eliminate the ability of the C1q binding peptide alone (without these molecules) to bind C1q and/or inhibit classical complement activation. A “C1q binding region” is any portion of the monoclonal antibodies or a functional variant thereof, provided herein, which specifically binds a human C1q epitope. Therefore, C1q binding peptides include any peptide that is able to specifically bind a human C1q epitope. A “human C1q epitope” as used herein is a portion of human C1q which is selectively bound by the C1q binding peptide provided herein. C1q binding peptides, which inhibit CCP activation are also referred to as C1q inhibitors.

The C1q binding peptide may be an antibody or a functionally active antibody fragment. For example, the invention provides, a monoclonal antibody that was found to bind human C1q and inhibit classical complement activation. The C1q binding peptide of the invention may be, for example, the deposited monoclonal antibody (mAb). The invention further provides a hybridoma cell line, which produces this monoclonal antibody. This hybridoma was deposited pursuant to, and in satisfaction of, the requirements of the Budapest Treaty on the International Recognition of the Deposit of Microorganisms for the Purposes of Patent Procedure with the American Type Culture Collection (“ATCC”) as an International Depository Authority and given the Patent Deposit Designations PTA-5226. For purposes of brevity the term “deposited hybridoma” or “hybridoma P1 H10” or “P1 H10” is used throughout the specification to refer to the hybridoma deposited with the ATCC on May 28, 2003. The term “deposited monoclonal antibody” or “P1 H10 anti-human C1q mAb” or “P1 H10 mAb” is used to refer to the monoclonal antibody produced by the ATCC deposited hybridoma.

Antibodies are well known to those of ordinary skill in the science of immunology. The C1q binding peptides include not only intact antibody molecules but also fragments of antibody molecules retaining C1q binding ability. C1q binding peptides provided herein are also peptides that contain a C1q binding region that is an antigen binding fragment. As used herein the term “antigen binding fragment” means a fragment of an antibody, which has the same C1q binding specificity as the deposited monoclonal antibody. Such fragments are also well known in the art and are regularly employed both in vitro and in vivo. In particular, as used herein, the term “C1q binding peptide” includes not only intact immunoglobulin molecules but also the well-known active fragments F(ab′)₂, Fab, Fd and Fv. F(ab′)₂, and Fab fragments or conservatively substituted versions thereof which lack the Fc fragment of intact antibody, clear more rapidly from the circulation, and may have less non-specific tissue binding of an intact antibody (Wahl et al., J. Nucl. Med. 24:316-325 (1983)). Single-chain antibodies can also be constructed in accordance with the methods described in U.S. Pat. No. 4,946,778 to Ladner et al. Such single-chain antibodies include the variable regions of the light and heavy chains joined by a flexible linker moiety. Methods for obtaining a single domain antibody (“Fd”) which comprises an isolated variable heavy chain single domain, also have been reported (see, for example, Ward et al., Nature 341:644-646 (1989), disclosing a method of screening to identify an antibody heavy chain variable region (V_{H} single domain antibody) with sufficient affinity for its target epitope to bind thereto in isolated form). Methods for making recombinant Fv fragments based on known antibody heavy chain and light chain variable region sequences are known in the art and have been described, e.g., Moore et al., U.S. Pat. No. 4,462,334. Other references describing the use and generation of antibody fragments include e.g., Fab fragments (Tijssen, Practice and Theory of Enzyme Immunoassays (Elsevieer, Amsterdam, 1985)), Fv fragments (Hochman et al., Biochemistry 12: 1130 (1973); Sharon et al., Biochemistry 15: 1591 (1976); Ehrilch et al., U.S. Pat. No. 4,355,023) and portions of antibody molecules (Audilore-Hargreaves, U.S. Pat. No. 4,470,925). Those skilled in the art may construct antibody fragments from various portions of intact antibodies without destroying the specificity of the antibodies for the C1q epitope.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The pFc′ and Fc regions of the antibody, for example, are effectors of the complement cascade but are not involved in antigen binding. An antibody from which the pFc′ region has been enzymatically cleaved, or which has been produced without the pFc′ region, designated an F(ab′)₂ fragment, retains both of the antigen binding sites of an intact antibody. An isolated F(ab′)₂ fragment is referred to as a bivalent monoclonal fragment because of its two antigen binding sites. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd (heavy chain variable region). The Fd fragments are the major determinant of antibody specificity (a single Fd fragment may be associated with up to ten different light chains without altering antibody specificity) and Fd fragments retain epitope-binding ability in isolation. The terms Fab, Fc, pFc′, F(ab′)2 and Fv are used consistently with their standard immunological meanings [Klein, Immunology (John Wiley, New York, N.Y., 1982); Clark, W. R. (1986) The Experimental Foundations of Modern Immunology (Wiley & Sons, Inc., New York); Roitt, I. (1991) Essential Immunology, 7th Ed., (Blackwell Scientific Publications, Oxford)].

The antigen binding fragments are also complementarity determining regions (CDRs) of the antibody. As is well-known in the art, the complementarity determining regions (CDRs) of an antibody are the portions of the antibody which are largely responsible for antibody specificity. The CDRs directly interact with the epitope of the antigen (see, in general, Clark, 1986; Roitt, 1991). In both the heavy chain and the light chain variable regions of IgG immunoglobulins, there are four framework regions (FR1 through FR4) separated respectively by three complementarity determining regions (CDR1 through CDR3). The framework regions (FRs) maintain the tertiary structure of the paratope, which is the portion of the antibody which is involved in the interaction with the antigen. The CDRs, include the light chain CDR1 and CDR2 regions, and in particular the CDR3 regions, and more particularly the heavy chain CDR3 contribute to antibody specificity. Because these CDR regions, and in particular the CDR3 region, confer antigen specificity on the antibody these regions may be incorporated into other antibodies or peptides to confer the identical antigen specificity onto that antibody or peptide.

The C1q binding region, therefore, can be an antigen-binding fragment, such as a CDR1, CDR2 or a CDR3 region or a combination thereof or conservatively substituted versions thereof, which binds C1q. For instance, a “C1q binding CDR3 region”, as used herein, is a CDR3 peptide sequence derived from the monoclonal antibodies produced by the hybridoma deposited with the ATCC Accession No. PTA-5226. A “C1q binding CDR2 region” is a CDR2 peptide sequence derived from the monoclonal antibodies produced by the deposited hybridoma, and, likewise, a “C1q binding CDR1 region” is a CDR1 peptide sequence derived from the monoclonal antibodies produced by the deposited hybridoma. As used herein, “CDR3_{P1 H10}” includes the CDR3 region of the P1 H10 anti-human C1q mAb, which specifically binds to C1q. A “CDR2_{P1 H10}” includes the CDR2 region of the P1 H10 anti-human C1q mAb, and a “CDR1_{P1 H10}” includes the CDR1 region of the P1 H10 anti-human C1q mAb. In some instances, C1q binding peptides with a CDR3_{P1 H10}, CDR2_{P1 H10}, or CDR1_{P1 H10} also prevent classical complement activation.

Antigen binding fragments also encompass “humanized antibody fragments.” As one skilled in the art will recognize, such fragments can be prepared by traditional enzymatic cleavage of intact humanized antibodies. If, however, intact antibodies are not susceptible to such cleavage, because of the nature of the construction involved, the noted constructions can be prepared with immunoglobulin fragments used as the starting materials; or, if recombinant techniques are used, the DNA sequences, themselves, can be tailored to encode the desired “fragment” which, when expressed, can be combined in vivo or in vitro, by chemical or biological means, to prepare the final desired intact immunoglobulin fragment.

C1q binding peptides also include peptides which are functional variants. A “functional variant” as used herein is a peptide having the sequence of the antibody or antigen-binding fragment region with conservative substitutions therein. As used herein, “conservative substitution” refers to an amino acid substitution which does not alter the relative charge or size characteristics of the peptide in which the amino acid substitution is made. Conservative substitutions of amino acids include substitutions made amongst amino acids with the following groups: (1) M,I,L,V; (2) F,Y,W; (3) K,R,H; (4) A,G; (5) S,T; (6) Q,N; and, (7) E,D. Conservative substitutions also include those fulfilling the criteria defined for an “accepted point mutation” in Dayhoff et al., 5: Atlas of Protein Sequence and Structure, 5: Suppl. 3, chapter 22: 354-352, Nat. Biomed. Res. Foundation, Washington, D.C. (1978). Such substitutions can be made by a variety of methods known to one of ordinary skill in the art. For example, amino-acid substitutions may be made by PCR-directed mutation, site-directed mutagenesis according to the method of Kunkel (Kunkel, Proc. Nat. Acad. Sci. U.S.A. 82: 488-492, 1985), or by chemical synthesis of a gene encoding the C1q binding peptide. These and other methods for altering a peptide will be known to those of ordinary skill in the art and may be found in references which compile such methods, e.g. Sambrook. et al., Molecular Cloning: A Laboratory Manual, 2nd edition, Cold Spring Harbor Laboratory Press, 1989. The activity of functionally equivalent variants of the C1q binding peptides can be tested by the binding and activity assays discussed herein. In some embodiments, the C1q binding peptides derived from the deposited monoclonal antibody or a fragment thereof contain 1, 2, 3, 4, 5, 6, 7, 8, 9, 10, 11, 12, 13, 14, 15, 16, 17, 18, 19, 20, 21, 22, 23, 24, 25, 30, 40, 50 or more substitutions.

The C1q binding peptides provided, including intact antibodies, have the binding characteristics of the deposited monoclonal antibody. A C1q binding peptide having the binding characteristics of the deposited monoclonal antibody is one which specifically (i.e., selectively) binds to the same epitopes of human C1q that the deposited antibody interacts with (i.e., defined by the deposited monoclonal antibody). In some embodiments, the C1q binding peptide with binding characteristics of the deposited monoclonal antibody is one that also inhibits classical complement activation. One of ordinary skill in the art can easily identify C1q binding peptides having the binding characteristics of the deposited monoclonal antibody using the screening and binding assays set forth in detail below.

To determine the epitope, one can use standard epitope mapping methods known in the art. For example, fragments (peptides) of C1q (preferably synthetic peptides) that bind the deposited monoclonal antibody can be used to determine whether a candidate C1q binding peptide binds the same epitope. For linear epitopes, overlapping peptides of a defined length (e.g., 8 or more amino acids) are synthesized. The peptides preferably are offset by 1 amino acid, such that a series of peptides covering every 8 amino acid fragment of the C1q protein sequence are prepared. Fewer peptides can be prepared by using larger offsets, e.g., 2 or 3 amino acids. In addition, longer peptides (e.g., 9-, 10- or 11-mers) can be synthesized. Binding of peptides to antibodies can be determined using standard methodologies including surface plasmon resonance (BIACORE) and ELISA assays. For examination of conformational epitopes, larger C1q fragments can be used. Other methods that use mass spectrometry to define conformational epitopes have been described and can be used (see, e.g., Baerga-Ortiz et al., Protein Science 11:1300-1308, 2002 and references cited therein). Still other methods for epitope determination are provided in standard laboratory reference works, such as Unit 6.8 (“Phage Display Selection and Analysis of B-cell Epitopes”) and Unit 9.8 (“Identification of Antigenic Determinants Using Synthetic Peptide Combinatorial Libraries”) of Current Protocols in Immunology, Coligan et al., eds., John Wiley & Sons. Epitopes can be confirmed by introducing point mutations or deletions into a known epitope, and then testing binding with the deposited monoclonal antibody or other C1q binding peptide.

The C1q binding peptide can also be one that competitively inhibits the specific binding of a second antibody to its target epitope on human C1q, wherein the second antibody is the deposited monoclonal antibody. To determine competitive inhibition, a variety of assays known to one of ordinary skill in the art can be employed. Screening of peptides to identify C1q binding peptides which competitively inhibit the binding of the deposited monoclonal antibody to C1q can be carried out utilizing a competition assay. If the peptide being tested competes with the deposited monoclonal antibody, as shown by a decrease in binding of the deposited monoclonal antibody, then it is likely that the peptide and the deposited monoclonal antibody bind to the same, or a closely related, epitope. Still another way to determine whether a peptide has the specificity of the deposited monoclonal antibody of the invention is to pre-incubate the deposited monoclonal antibody with C1q with which it is normally reactive, and then add the peptide being tested to determine if the peptide being tested is inhibited in its ability to bind C1q. If the peptide being tested is inhibited then, in all likelihood, it has the same, or a functionally equivalent, epitope and specificity as the deposited monoclonal antibody. Other assays that evaluate the ability of antibodies to cross-compete for C1q molecules, in solid phase or in solution phase can be used.

Preferred C1q binding peptides competitively inhibit the specific binding of a second antibody to its target epitope on C1q by at least about 10%, 20%, 30%, 40%, 50%, 60%, 70%, 80%, 90%, 95%, or 99%. Inhibition can be assessed at various molar ratios or mass ratios; for example competitive binding experiments can be conducted with a 2-fold, 3-fold, 4-fold, 5-fold, 7-fold, 10-fold or more molar excess of the C1q binding peptide over the deposited monoclonal antibody.

By using the deposited monoclonal antibodies of the invention, it is also possible to produce anti-idiotypic antibodies which can be used to screen other antibodies to identify whether the antibody has the same binding specificity as the deposited monoclonal antibodies of the invention. In addition, such anti-idiotypic antibodies can be used for active immunization (Herlyn, et al., Science, 232:100, 1986). Such anti-idiotypic antibodies can be produced using well-known hybridoma techniques (Kohler and Milstein, Nature, 256:495, 1975). An anti-idiotypic antibody is an antibody which recognizes unique determinants present on the deposited monoclonal antibody. These determinants are located in the hypervariable region of the antibody. It is this region which binds to a given epitope and, thus, is responsible for the specificity of the antibody. An anti-idiotypic antibody can be prepared by immunizing an animal with the deposited monoclonal antibodies. The immunized animal will recognize and respond to the idiotypic determinants of the immunizing deposited monoclonal antibodies and produce an antibody to these idiotypic determinants. By using the anti-idiotypic antibodies of the immunized animal, which are specific for the deposited monoclonal antibodies of the invention, it is possible to identify other clones with the same idiotype as the deposited monoclonal antibody used for immunization. Idiotypic identity between monoclonal antibodies of two cell lines demonstrates that the two monoclonal antibodies are the same with respect to their recognition of the same epitopic determinant. Thus, by using anti-idiotypic antibodies, it is possible to identify other hybridomas expressing monoclonal antibodies having the same epitopic specificity.

It is also possible to use the anti-idiotype technology to produce monoclonal antibodies which mimic an epitope. For example, an anti-idiotypic monoclonal antibody made to a first monoclonal antibody will have a binding domain in the hypervariable region which is the image of the epitope bound by the first monoclonal antibody. Thus, the anti-idiotypic monoclonal antibody can be used for immunization, since the anti-idiotype monoclonal antibody binding domain effectively acts as an antigen.

In one set of embodiments, the peptide useful according to the methods of the present invention is an intact humanized anti-C1q monoclonal antibody in an isolated form or in a pharmaceutical preparation. The following examples of methods for preparing humanized monoclonal antibodies that interact with C1q and/or inhibit classical complement activation are exemplary and are provided for illustrative purposes only.

A “humanized monoclonal antibody” as used herein is a human monoclonal antibody or functionally active fragment thereof having human constant regions and a C1q binding CDR region (e.g. a CDR3 region) from a mammal of a species other than a human. Humanized monoclonal antibodies may be made by any method known in the art. Humanized monoclonal antibodies, for example, may be constructed by replacing the non-CDR regions of a non-human mammalian antibody with similar regions of human antibodies while retaining the epitopic specificity of the original antibody. For example, non-human CDRs and optionally some of the framework regions may be covalently joined to human FR and/or Fc/pFc′ regions to produce a functional antibody. There are entities in the United States which will synthesize humanized antibodies from specific murine antibody regions commercially, such as Protein Design Labs (Mountain View Calif.).

European Patent Application 0239400, the entire contents of which is hereby incorporated by reference, provides an exemplary teaching of the production and use of humanized monoclonal antibodies in which at least the CDR portion of a murine (or other non-human mammal) antibody is included in the humanized antibody. Briefly, the following methods are useful for constructing a humanized CDR monoclonal antibody including at least a portion of a mouse CDR. A first replicable expression vector including a suitable promoter operably linked to a DNA sequence encoding at least a variable domain of an Ig heavy or light chain and the variable domain comprising framework regions from a human antibody and a CDR region of a murine antibody is prepared. Optionally a second replicable expression vector is prepared which includes a suitable promoter operably linked to a DNA sequence encoding at least the variable domain of a complementary human Ig light or heavy chain respectively. A cell line is then transformed with the vectors. Preferably the cell line is an immortalized mammalian cell line of lymphoid origin, such as a myeloma, hybridoma, trioma, or quadroma cell line, or is a normal lymphoid cell which has been immortalized by transformation with a virus. The transformed cell line is then cultured under conditions known to those of skill in the art to produce the humanized antibody.

As set forth in European Patent Application 0239400 several techniques are well known in the art for creating the particular antibody domains to be inserted into the replicable vector. (Preferred vectors and recombinant techniques are discussed in greater detail below.) For example, the DNA sequence encoding the domain may be prepared by oligonucleotide synthesis. Alternatively a synthetic gene lacking the CDR regions in which four framework regions are fused together with suitable restriction sites at the junctions, such that double stranded synthetic or restricted subcloned CDR cassettes with sticky ends could be ligated at the junctions of the framework regions. Another method involves the preparation of the DNA sequence encoding the variable CDR containing domain by oligonucleotide site-directed mutagenesis. Each of these methods is well known in the art. Therefore, those skilled in the art may construct humanized antibodies containing a murine CDR region without destroying the specificity of the antibody for its epitope.

In preferred embodiments, the humanized antibodies of the invention are human monoclonal antibodies including at least the C1q binding CDR3 region of the deposited monoclonal antibody. As noted above, such humanized antibodies may be produced in which some or all of the FR regions of deposited monoclonal antibodies have been replaced by homologous human FR regions. In addition, the Fc portions may be replaced so as to produce IgA or IgM as well as human IgG antibodies bearing some or all of the CDRs of the deposited monoclonal antibody. Of particular importance is the inclusion of the deposited monoclonal antibody C1q binding CDR3 region and, to a lesser extent, the other CDRs and portions of the framework regions of the deposited monoclonal antibody. Such humanized antibodies will have particular clinical utility in that they will specifically recognize human C1q but will not evoke an immune response in humans against the antibody itself. In a most preferred embodiment, a murine CDR is grafted into the framework region of a human antibody to prepare the “humanized antibody.” See, e.g., L. Riechmann et al., Nature 332, 323 (1988); M. S. Neuberger et al., Nature 314, 268 (1985) and EPA 0 239 400 (published Sep. 30, 1987).

C1q binding peptides may easily be synthesized or produced by recombinant means by those of skill in the art. Methods for preparing or identifying peptides which bind to a particular target are well known in the art. Molecular imprinting, for instance, may be used for the de novo construction of macromolecular structures such as peptides which bind to a particular molecule. See for example Kenneth J. Shea, Molecular Imprinting of Synthetic Network Polymers: The De Novo synthesis of Macromolecular Binding and Catalytic Sites, TRIP Vol. 2, No. 5, May 1994; Klaus Mosbach, Molecular Imprinting, Trends in Biochem. Sci., 19(9) January 1994; and Wulff, G., in Polymeric Reagents and Catalysts (Ford, W. T., Ed.) ACS Symposium Series No. 308, pp 186-230, American Chemical Society (1986). One method for preparing mimics of C1q binding peptides involves the steps of: (i) polymerization of functional monomers around a known C1q binding peptide or the binding region of an anti-C1q antibody (such as the deposited antibody) (the template) that exhibits a desired activity; (ii) removal of the template molecule; and then (iii) polymerization of a second class of monomers in the void left by the template, to provide a new molecule which exhibits one or more desired properties which are similar to that of the template. In addition to preparing peptides in this manner other C1q binding molecules which are C1q inhibitors such as polysaccharides, nucleosides, drugs, nucleoproteins, lipoproteins, carbohydrates, glycoproteins, steroids, lipids, and other biologically active materials can also be prepared. This method is useful for designing a wide variety of biological mimics that are more stable than their natural counterparts, because they are typically prepared by the free radical polymerization of functional monomers, resulting in a compound with a nonbiodegradable backbone. Other methods for designing such molecules include for example drug design based on structure activity relationships which require the synthesis and evaluation of a number of compounds and molecular modeling.

The sequence of the CDR regions, for instance, for use in synthesizing peptides of the invention, may be determined by methods known in the art. The heavy chain variable region is a peptide which generally ranges from 100 to 150 amino acids in length. The light chain variable region is a peptide which generally ranges from 80 to 130 amino acids in length. The CDR sequences within the heavy and light chain variable regions which include only approximately 3-25 amino acid sequences may easily be sequenced by one of ordinary skill in the art. The peptides may even be synthesized by commercial sources such as by the Scripps Protein and Nucleic Acids Core Sequencing Facility (La Jolla Calif.).

The peptides can also be produced by recombinant techniques by incorporating the DNA expressing the peptide into an expression vector and transforming cells with the expression vector to produce the peptide. The sequences responsible for the specificity of the deposited monoclonal antibody can easily be determined by one of ordinary skill in the art so that peptides according to the invention can be prepared using recombinant DNA technology. There are entities in the United States which will perform this function commercially, such as Thomas Jefferson University and the Scripps Protein and Nucleic Acids Core Sequencing Facility (La Jolla Calif.). For example, the variable region cDNA can be prepared by polymerase chain reaction from the deposited hybridoma RNA using degenerate or non-degenerate primers (derived from the amino acid sequence). The cDNA can be subcloned to produce sufficient quantities of double stranded DNA for sequencing by conventional sequencing reactions or equipment.

Once the nucleic acid sequences of the heavy chain Fd and light chain variable domains of the deposited C1q monoclonal antibody are determined, one of ordinary skill in the art is now enabled to produce nucleic acids which encode this antibody or which encode the various antibody fragments, humanized antibodies, or peptides described above. It is contemplated that such nucleic acids will be operably joined to other nucleic acids forming a recombinant vector for cloning or for expression of the peptides of the invention. The present invention includes any recombinant vector containing the coding sequences, or part thereof, whether for prokaryotic or eukaryotic transformation, transfection or gene therapy. Such vectors may be prepared using conventional molecular biology techniques, known to those with skill in the art, and would comprise DNA coding sequences for the CDR3 region and additional variable sequences contributing to the specificity of the antibodies or parts thereof, as well as other non-specific peptide sequences and a suitable promoter either with (Whittle et al., Protein Eng. 1:499, 1987 and Burton et al., Science 266:1024-1027, 1994) or without (Marasco et al., Proc. Natl. Acad. Sci. (USA) 90:7889, 1993 and Duan et al., Proc. Natl. Acad. Sci. (USA) 91:5075-5079,1994) a signal sequence for export or secretion. Such vectors may be transformed or transfected into prokaryotic (Huse et al., Science 246:1275, 1989, Ward et al., Nature 341: 644-646, 1989; Marks et al., J. Mol. Biol. 222:581, 1991 and Barbas et al., Proc. Natl. Acad. Sci. (USA) 88:7978, 991) or eukaryotic (Whittle et al., 1987 and Burton et al., 1994) cells or used for gene therapy (Marasco et al., 1993 and Duan et al., 1994) by conventional techniques, known to those with skill in the art.

As used herein, a “vector” may be any of a number of nucleic acids into which a desired sequence may be inserted by restriction and ligation for transport between different genetic environments or for expression in a host cell. Vectors are typically composed of DNA although RNA vectors are also available. Vectors include, but are not limited to, plasmids and phagemids. A cloning vector is one which is able to replicate in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host bacterium or just a single time per host before the host reproduces by mitosis. In the case of phage, replication may occur actively during a lytic phase or passively during a lysogenic phase. An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase or alkaline phosphatase), and genes which visibly affect the phenotype of transformed or transfected cells, hosts, colonies or plaques. Preferred vectors are those capable of autonomous replication and expression of the structural gene products present in the DNA segments to which they are operably joined.

The expression vectors of the present invention include regulatory sequences operably joined to a nucleotide sequence encoding one of the peptides of the invention. As used herein, the term “regulatory sequences” means nucleotide sequences which are necessary for or conducive to the transcription of a nucleotide sequence which encodes a desired peptide and/or which are necessary for or conducive to the translation of the resulting transcript into the desired peptide. Regulatory sequences include, but are not limited to, 5′ sequences such as operators, promoters and ribosome binding sequences, and 3′ sequences such as polyadenylation signals. The vectors of the invention may optionally include 5′ leader or signal sequences, 5′ or 3′ sequences encoding fusion products to aid in protein purification, and various markers which aid in the identification or selection of transformants. The choice and design of an appropriate vector is within the ability and discretion of one of ordinary skill in the art. The subsequent purification of the peptides may be accomplished by any of a variety of standard means known in the art.

A preferred vector for screening peptides, but not necessarily preferred for the mass production of the peptides of the invention, is a recombinant DNA molecule containing a nucleotide sequence that codes for and is capable of expressing a fusion peptide containing, in the direction of amino- to carboxy-terminus, (1) a prokaryotic secretion signal domain, (2) a peptide of the invention, and, optionally, (3) a fusion protein domain. The vector includes DNA regulatory sequences for expressing the fusion peptide, preferably prokaryotic regulatory sequences. Such vectors can be constructed by those with skill in the art and have been described by Smith et al. (Science 228:1315-1317, 1985), Clackson et al. (Nature 352:624-628, 1991); Kang et al. (in “Methods: A Companion to Methods in Enzymology: Vol. 2”, R. A. Lerner and D. R. Burton, ed. Academic Press, NY, pp 111-118,1991); Barbas et al. (Proc. Natl. Acad. Sci. (USA) 88:7978-7982, 1991), Roberts et al. (Proc. Natl. Acad Sci. (USA) 89:2429-2433, 1992)

A fusion peptide may be useful for purification of the peptides of the invention. The fusion domain may, for example, include a poly-His tail which allows for purification on Ni+ columns or the maltose binding protein of the commercially available vector pMAL (New England BioLabs, Beverly, Mass.). A currently preferred, but by no means necessary, fusion domain is a filamentous phage membrane anchor. This domain is particularly useful for screening phage display libraries of monoclonal antibodies but may be of less utility for the mass production of antibodies. The filamentous phage membrane anchor is preferably a domain of the cpIII or cpVIII coat protein capable of associating with the matrix of a filamentous phage particle, thereby incorporating the fusion peptide onto the phage surface, to enable solid phase binding to specific antigens or epitopes and thereby allow enrichment and selection of the specific antibodies or fragments encoded by the phagemid vector.

The secretion signal is a leader peptide domain of a protein that targets the protein membrane of the host cell, such as the periplasmic membrane of gram negative bacteria. A preferred secretion signal for E. coli is a pelB secretion signal. The predicted amino acid residue sequences of the secretion signal domain from two pelB gene producing variants from Erwinia carotova are described in Lei, et al. (Nature 381:543-546, 1988). The leader sequence of the pelB protein has previously been used as a secretion signal for fusion proteins (Better, et al., Science 240:1041-1043, 1988; Sastry, et al., Proc. Natl. Acad. Sci (USA) 86:5728-5732, 1989; and Mullinax, et al., Proc. Natl. Acad. Sci. (USA) 87:8095-8099, 1990). Amino acid residue sequences for other secretion signal peptide domains from E. coli useful in this invention can be found in Oliver, In Neidhard, F. C. (ed.), Escherichia coli and Salmonella Typhimurium, American Society for Microbiology, Washington, D.C., 1:56-69 (1987).

To achieve high levels of gene expression in E. coli, it is necessary to use not only strong promoters to generate large quantities of mRNA, but also ribosome binding sites to ensure that the mRNA is efficiently translated. In E. coli, the ribosome binding site includes an initiation codon (AUG) and a sequence 3-9 nucleotides long located 3-11 nucleotides upstream from the initiation codon (Shine, et al., Nature 254:34, 1975). The sequence, AGGAGGU, which is called the Shine-Dalgarno (SD) sequence, is complementary to the 3′ end of E. coli 16S rRNA. Binding of the ribosome to mRNA and the sequence at the 3′ end of the mRNA can be affected by several factors:
(i) The degree of complementarity between the SD sequence and 3′ end of the 16S rRNA.
(ii) The spacing and possibly the DNA sequence lying between the SD sequence and the AUG (Roberts, et al., Proc. Natl. Acad. Sci. (USA) 76:760.,1979a: Roberts, et al., Proc. Natl. Acad. Sci. (USA) 76:5596, 1979b; Guarente, et al., Science 209:1428, 1980; and Guarente, et al., Cell 20:543, 1980). Optimization is achieved by measuring the level of expression of genes in plasmids in which this spacing is systematically altered. Comparison of different mRNAs shows that there are statistically preferred sequences from positions −20 to +13 (where the A of the AUG is position 0) (Gold, et al., Annu. Rev. Microbiol. 35:365, 1981). Leader sequences have been shown to influence translation dramatically (Roberts, et al., 1979a, b supra).
(iii) The nucleotide sequence following the AUG, which affects ribosome binding (Taniguchi, et al., J. Mol. Biol., 118:533, 1978).
   The 3′ regulatory sequences define at least one termination (stop) codon in frame with and operably joined to the heterologous fusion peptide.

In preferred embodiments with a prokaryotic expression host, the vector utilized includes a prokaryotic origin of replication or replicon, i.e., a DNA sequence having the ability to direct autonomous replication and maintenance of the recombinant DNA molecule extra-chromosomally in a prokaryotic host cell, such as a bacterial host cell, transformed therewith. Such origins of replication are well known in the art. Preferred origins of replication are those that are efficient in the host organism. A preferred host cell is E. coli. For use of a vector in E. coli, a preferred origin of replication is ColE1 found in pBR322 and a variety of other common plasmids. Also preferred is the p15A origin of replication found on pACYC and its derivatives. The ColE1 and p15A replicons have been extensively utilized in molecular biology, are available on a variety of plasmids and are described by Sambrook. et al., Molecular Cloning: A Laboratory Manual, 2nd edition, Cold Spring Harbor Laboratory Press, 1989).

In addition, those embodiments that include a prokaryotic replicon preferably also include a gene whose expression confers a selective advantage, such as drug resistance, to a bacterial host transformed therewith. Typical bacterial drug resistance genes are those that confer resistance to ampicillin, tetracycline, neomycin/kanamycin or chloramphenicol. Vectors typically also contain convenient restriction sites for insertion of translatable DNA sequences. Exemplary vectors are the plasmids pUC18 and pUC19 and derived vectors such as pcDNAII available from Invitrogen, (San Diego, Calif.).

When the peptide of the invention is an antibody including both heavy chain and light chain sequences, these sequences may be encoded on separate vectors or, more conveniently, may be expressed by a single vector. The heavy and light chain may, after translation or after secretion, form the heterodimeric structure of natural antibody molecules. Such a heterodimeric antibody may or may not be stabilized by disulfide bonds between the heavy and light chains.

A vector for expression of heterodimeric antibodies, such as the intact antibodies of the invention or the F(ab′)₂, Fab, Fd or Fv fragment antibodies of the invention, is a recombinant DNA molecule adapted for receiving and expressing translatable first and second DNA sequences. That is, a DNA expression vector for expressing a heterodimeric antibody provides a system for independently cloning (inserting) the two translatable DNA sequences into two separate cassettes present in the vector, to form two separate cistrons for expressing the first and second peptides of a heterodimeric antibody. The DNA expression vector for expressing two cistrons is referred to as a dicistronic expression vector.

Preferably, the vector comprises a first cassette that includes upstream and downstream DNA regulatory sequences operably joined via a sequence of nucleotides adapted for directional ligation to an insert DNA. The upstream translatable sequence preferably encodes the secretion signal as described above. The cassette includes DNA regulatory sequences for expressing the first antibody peptide that is produced when an insert translatable DNA sequence (insert DNA) is directionally inserted into the cassette via the sequence of nucleotides adapted for directional ligation.

The dicistronic expression vector also contains a second cassette for expressing the second antibody peptide. The second cassette includes a second translatable DNA sequence that preferably encodes a secretion signal, as described above, operably joined at its 3′ terminus via a sequence of nucleotides adapted for directional ligation to a downstream DNA sequence of the vector that typically defines at least one stop codon in the reading frame of the cassette. The second translatable DNA sequence is operably joined at its 5′ terminus to DNA regulatory sequences forming the 5′ elements. The second cassette is capable, upon insertion of a translatable DNA sequence (insert DNA), of expressing the second fusion peptide comprising a secretion signal with a peptide coded by the insert DNA.

The peptides of the present invention may also, of course, be produced by eukaryotic cells such as CHO cells, human hybridomas, immortalized B-lymphoblastoid cells, and the like. In this case, a vector is constructed in which eukaryotic regulatory sequences are operably joined to the nucleotide sequences encoding the peptide. The design and selection of an appropriate eukaryotic vector is within the ability and discretion of one of ordinary skill in the art. The subsequent purification of the peptides may be accomplished by any of a variety of standard means known in the art.

In addition host cells are provided, both prokaryotic and eukaryotic, transformed or transfected with, and therefore including, the vectors of the present invention.

As used herein, a coding sequence and regulatory sequences are said to be “operably joined” when they are covalently linked in such a way as to place the expression or transcription of the coding sequence under the influence or control of the regulatory sequences. If it is desired that the coding sequences be translated into a functional peptide, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript might be translated into the desired peptide.

The precise nature of the regulatory sequences needed for gene expression may vary between species or cell types, but shall in general include, as necessary, 5′ non-transcribing and 5′ non-translating sequences involved with initiation of transcription and translation respectively, such as a TATA box, capping sequence, CAAT sequence, and the like. Especially, such 5′ non-transcribing regulatory sequences will include a promoter region which includes a promoter sequence for transcriptional control of the operably joined gene. Regulatory sequences may also include enhancer sequences or upstream activator sequences, as desired.

With the sequence of the deposited antibody or antigen-binding fragments thereof, one of skill in the art is also enabled to identify other C1q binding peptides encoded by nucleic acid molecules that are homologous to the sequence of the deposited monoclonal antibody or antigen-binding fragments thereof. Preferably the homologous nucleic acid molecule comprises a nucleotide sequence that is at least about 90% identical to the nucleotide sequence provided herein. More preferably, the nucleotide sequence is at least about 95% identical, at least about 97% identical, at least about 98% identical, or at least about 99% identical to the nucleotide sequence provided herein. The homology can be calculated using various, publicly available software tools well known to one of ordinary skill in the art. Exemplary tools include the BLAST system available from the website of the National Center for Biotechnology Information (NCBI) at the National Institutes of Health.

One method of identifying highly homologous nucleotide sequences is via nucleic acid hybridization. Thus the invention also includes C1q binding peptides having the C1q binding properties and other functional properties described herein, which are encoded by nucleic acid molecules that hybridize under high stringency conditions to the foregoing nucleic acid molecules. Identification of related sequences can also be achieved using polymerase chain reaction (PCR) and other amplification techniques suitable for cloning related nucleic acid sequences. Preferably, PCR primers are selected to amplify portions of a nucleic acid sequence of interest, such as a CDR.

The term “high stringency conditions” as used herein refers to parameters with which the art is familiar. Nucleic acid hybridization parameters may be found in references that compile such methods, e.g. Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. One example of high-stringency conditions is hybridization at 65° C. in hybridization buffer (3.5×SSC, 0.02% Ficoll, 0.02% polyvinyl pyrrolidone, 0.02% Bovine Serum Albumin, 2.5 mM NaH₂PO₄(pH7), 0.5% SDS, 2 mM EDTA). SSC is 0.15M sodium chloride/0.015M sodium citrate, pH7; SDS is sodium dodecyl sulphate; and EDTA is ethylenediaminetetracetic acid. After hybridization, a membrane upon which the nucleic acid is transferred is washed, for example, in 2×SSC at room temperature and then at 0.1-0.5×SSC/0.1×SDS at temperatures up to 68° C.

Other C1q binding peptides include peptides with an amino acid sequence that is at least 50%, 60%, 70%, 75%, 80%, 85%, 90%, 95%, 97% or 99% identical to the amino acid sequence of the deposited monoclonal antibody or a fragment thereof.

C1q binding peptides can also be identified using routine assays, such as with binding and complement activation assays. The C1q binding peptides as described herein can be tested for their ability to bind C1q using routine in vitro binding assays. The ability of these peptides to block C1q deposition or to prevent its association with other classical complement components, thereby blocking classical complement activation, can be detected using functional activity assays.

To determine whether a peptide binds to C1q, any known binding assay may be employed. For example, the peptide may be immobilized on a surface and then contacted with a labeled C1q. The amount of C1q which interacts with the peptide or the amount which does not bind to the peptide may then be quantitated to determine whether the peptide binds to C1q. A surface having the deposited monoclonal antibody immobilized thereto may serve as a positive control.

Using routine procedures known to those of ordinary skill in the art, one can determine whether a peptide which binds to C1q is also one which blocks C1q from binding to a C1q ligand and inhibits classical complement activation.

Functional activity of the classical complement pathway may be assessed by ELISA using human IgM as a ligand. Nunc Maxisorb plates (Nunc, Roskilde, Denmark) are coated with human IgM at 2 μg/ml in coating buffer (100 mM Na₂CO₃/NaHCO₃, pH 9.6), for 16 hours at room temperature or for 2 hours at 37° C. After each step, plates are washed three times with PBS containing 0.05% Tween 20. Residual binding sites are blocked by incubation with PBS containing 1% BSA for one hour at 37° C. After blocking of residual binding sites, serum samples, diluted in GVB++, are added to the plate and incubated for 1 hour at 37° C. Complement binding may be assessed using dig-conjugated mAb directed against C1q, C4, C3, and C5b-9, followed by the detection of mAb binding using HRP-conjugated sheep anti-digoxygenin antibodies. All detection antibodies are diluted in PBS containing 1% BSA and 0.05% Tween 20. Enzyme activity of HRP is detected following incubation with 2,2′-azino-bis(3-ethylbenzthiazoline-6-sulfonic acid) (from Sigma; 2.5 mg/ml in 0.1 M Citrate/Na₂HPO₄ buffer, pH 4.2) in the presence of 0.01% H₂O₂, for 30-60 min. at room temperature. The OD at 415 nm is measured using a microplate biokinetics reader (EL312e, from Biotek Instruments, Winooski, Vt.).

Functional activity can also be assessed with a variety of experimental methods which include using sensitized chicken red blood cells (RBCs), Western analysis, pull down experiments followed by Western analysis, etc.

Activation assays also can be used to assess the relative inhibitory concentrations of a peptide and to identify those peptides which inhibit classical complement activation by at least, e.g., 75%.

Other assays will be apparent to those of skill in the art, having read the present specification, which are useful for determining whether a peptide which binds to C1q also inhibits classical complement activation.

Still other peptides which bind to the C1q may also be identified by conventional screening methods such as phage display procedures (e.g., methods described in Hart, et al., J. Biol. Chem. 269:12468 (1994)). Hart et al. report a filamentous phage display library for identifying novel peptide ligands for mammalian cell receptors. In general, phage display libraries using, e.g., M13 or fd phage, are prepared using conventional procedures such as those described in the foregoing reference. The libraries display inserts containing from 4 to 80 amino acid residues. The inserts optionally represent a completely degenerate or a biased array of peptides. Ligands that bind selectively to C1q are obtained by selecting those phages which express on their surface a ligand that binds to the C1q. These phages then are subjected to several cycles of reselection to identify the peptide ligand-expressing phages that have the most useful binding characteristics. Typically, phages that exhibit the best binding characteristics (e.g., highest affinity) are further characterized by nucleic acid analysis to identify the particular amino acid sequences of the peptides expressed on the phage surface and the optimum length of the expressed peptide to achieve optimum binding to the C1q. Alternatively, such peptide ligands can be selected from combinatorial libraries of peptides containing one or more amino acids. Such libraries can further be synthesized which contain non-peptide synthetic moieties which are less subject to enzymatic degradation compared to their naturally-occurring counterparts.

In addition to the deposited monoclonal antibody, other antibodies (e.g., anti-C1q) can be generated. The following is a description of a method for developing a monoclonal antibody specific for C1q. The description is exemplary and is provided for illustrative purposes only.

Murine monoclonal antibodies may be made by any of the methods known in the art utilizing C1q as an immunogen. An example of a method for producing murine monoclonals useful according to the invention is the following: Female Balb/C mice are initially inoculated (i.p.) with 250 ul of the following mixture: 250 μl Titermax mixed with 100 μg human C1q in 250 μl PBS. The following week and for three consecutive weeks the mice are injected with 50 μg hC1q in 250 μlPBS. On the 4th week the mice are injected with 25 μg C1q in 250 μl PBS and the mice are fused 4 days later.

The fusion protocol is adapted from Current Protocols in Immunology. The splenocytes are fused 1:1 with myelinoma fusion partner P301 from ATCC using PEG 150 at 50% w/v. The fused cells are plated at a density of 1.25×10⁶/m. with 100 μl/well of a 96 well microtiter plate. The fusion media consists of Deficient DME high glucose, Pen/Strep (50,000 U pen, 50,000 82 g strep per liter), 4 mM L-glutamine, 20% fetal bovine serum, 10% thyroid enriched media, 1% OPI, 1% NEAA, 1% HAT, and 50 μM 2-mercaptoethanol. The cells are fed 100 μl/well on day one and 100/well media are exchanged on days 2, 3, 4, 7, 9, 11, and 13. The last media change before primary screening consists of HAT substituted for the 1% HT. All subsequent feedings are done with fusion media minus the minus HT or HAT. Screening is done with human C1q plated to plastic ELISA plates (96 well plates). Purified hC1q is plated in each well at 50 μl volume containing 2 μg/ml C1q in 2% sodium carbonate buffer. The plates are then blocked with 3% BSA in PBS. Tissue culture media (50 μl) is placed in the wells and incubated for 1 hour at room temperature. The plates are washed and a secondary HRP labeled goat anti-mouse IgG antibody is used for detection. Colorimetric analysis is done with ABTS and read at 405 nm. Positive controls consist of a polyclonal antibody to human C1q. Cells are then grown in media consisting of the following: DMEM high glucose no-I-glut, sod, pyruvate 500 ml (Irvine Scientific #9024), heat inactivated Hyclone 10%, 1% Non-essential amino acids, 4 mM L-glutamine, 100 U/ml penicillin and 100 μg/ml streptomycin. All positive wells are then screened for function in a secondary screen.

Human monoclonal antibodies may be made by any of the methods known in the art, such as those disclosed in U.S. Pat. No. 5,567,610, issued to Borrebaeck et al., U.S. Pat. No. 565,354, issued to Ostberg, U.S. Pat. No. 5,571,893, issued to Baker et al, Kozber, J. Immunol. 133: 3001 (1984), Brodeur, et al., Monoclonal Antibody Production Techniques and Applications, p. 51-63 (Marcel Dekker, Inc, new York, 1987), and Boerner el al., J. Immunol., 147: 86-95 (1991). In addition to the conventional methods for preparing human monoclonal antibodies, such antibodies may also be prepared by immunizing transgenic animals that are capable of producing human antibodies (e.g., Jakobovits et al., PNAS USA, 90: 2551 (1993), Jakobovits et al., Nature, 362: 255-258 (1993), Bruggermann et al., Year in Immuno., 7:33 (1993) and U.S. Pat. No. 5,569,825 issued to Lonberg).

An example of one method for producing human monoclonals useful according to the invention is the following: Peripheral Blood Lymphocytes (PBL) are isolated from healthy human donors using density centrifugation, and further separated into B, T and accessory (A) cells, described methods such as (Danielsson, L., Moller, S. A. & Borrebaeck, C. A. K. Immunology 61, 51-55 (1987)). PBL are fractionated into T and non-T cells by resetting with 2-amino ethyl (isothiouronium bromide)—treated sheep red corpuscles, and the latter cell population is incubated on Petri dishes coated with fibronectin or autologous plasma. Non-adherent cells (B-cells) are decanted, and adherent cells (accessory cells) are removed by 10 mM EDTA. The B cells are stimulated with 50 μg Staphylococcus aureus Cowan I/ml and irradiated (2000R) T cells with 10 μg PWM/ml overnight. The accessory cells are stimulated with 5 IU gamma interferon/ml and 10 μm indomethacin. The cell populations are cultured in supplemented RPMI 1640 which contains 10% human AB serum at a cell ratio of 2:1:0.4 (Ti:B:A) for a total of 6 days. The antigenic dose of C1q is 1 μg/ml. The culture is supplemented with recombinant IL-2 (5 U/ml) and sPWM-T (25% by vol.), produced by described methods such as (Danielsson, L., Moller, S. A. & Borrebaeck C. A. K. Immunology 61, 51-55 (1987)). T cells (10 cells/ml) suspended in serum-free RPMI 1640 are incubated with 2.5 mM freshly prepared Leu-OMe for 40 min at room temperature. The cells are then washed 3 times in RPMI 1640 containing 2% human antibody serum. The antibodies produced may be humanized as described above.

There are a variety of clinical uses for the C1q binding peptides provided. The C1q inhibitors, provided herein, can be used for inhibiting classical complement activation. In certain inflammatory conditions, including but not limited to autoimmune diseases, the classical pathway is pro-inflammatory, rather than anti-inflammatory, and the inhibition of C1q would decrease the inflammation and tissue injury. The C1q inhibitors can be used in methods to treat any classical complement mediated disorder in a subject.

As used herein, a subject is a mammal (e.g., human, non-human primate, cow, horse, pig, sheep, goat, dog, cat or rodent). In all embodiments, human subjects are preferred.

Methods of treatment of persons with or at risk of a classical complement mediated disorder are provided. A “person at risk of a disorder” is one who has a high probability of developing the disorder. These subjects include, for instance, subjects having a genetic abnormality, the presence of which has been demonstrated to have a correlative relation to a higher likelihood of developing the disorder and subjects exposed to agents that can cause the disorder. For instance, in the case of cancer, cancer causing agents are tobacco, asbestos, or other chemical toxins. A person at risk of a disorder is also one who previously have been treated for the disorder and are not presenting symptoms of the disorder (e.g., are in apparent remission). A “person with a disorder” is one who has been diagnosed with the disorder or who could be diagnosed as having the disorder. In the example where the disorder is a cancer, a person with cancer is one who has detectable cancer cells. As used herein the terms “treat” or “treating” can mean the reduction of symptoms associated with a specific disorder. These terms can also mean the elimination of a disorder or the inhibition in the progression of a disorder. These terms are also intended to include the use of a therapeutic to prevent the onset of the disorder in a subject that does not yet have the disorder. C1q inhibitors can be administered to a person with or at risk of a disorder in an effective amount to treat the disorder. C1q inhibitors can be provided alone or in combination with other therapeutic agents.

A “classical complement mediated disorder” as used herein is a disorder which involves inflammation and/or cellular injury caused by classical complement activation. Included in inflammation is inflammation that results from the administration of a therapeutic agent. The inflammation can produce unwanted side effects such as allergic reactions. The use of the term “inflammation” is intended to encompass unwanted side effects, such as allergic reactions, which can be reduced or eliminated with the administration of a classical complement inhibitor, such as a C1q inhibitor. Classical complement mediated disorders also include autoimmune disease. Preferably, the autoimmune disease is not arthritis (e.g., rheumatoid arthritis, collagen-induced arthritis), glomerulo-nephritis, lupus (e.g., system lupus erythematosus (SLE)), membranous nephropathy, or myasthenia gravis. Specific C1q disorders also include, but are not limited to, atherosclerosis, arthritis, ischemia and reperfusion, transplantation, cardiopulmonary bypass (CPB), stroke, acute respiratory distress syndrome (ARDS), lupus (e.g., SLE), Alzheimer's or dialysis. In some instances the disorder is not Alzheimer's. Each of these disorders is well-known in the art and/or is described, for instance, in Harrison 's Principles of Internal Medicine (McGraw Hill, Inc., New York).

In regard to ischemia and reperfusion, methods of treatment with the C1q inhibitors described herein are provided. Such methods of treatment include treatment of ischemia and reperfusion that occurs in the brain, lungs, kidneys and skeletal muscle. Methods of treatment of cardiac and gastrointestinal ischemia/reperfusion are not provided. In some embodiments the methods provided herein do not include ischemia reperfusion of the liver. In other embodiments the ischemia reperfusion is not hind limb ischemia reperfusion. In still other embodiments the ischemia reperfusion is not renal ischemia reperfusion.

“Inflammation” as used herein, is a localised protective response elicited by a foreign (non-self) antigen, and/or by an injury or destruction of tissue(s), which serves to destroy, dilute or sequester the foreign antigen, the injurious agent, and/or the injured tissue. Inflammation occurs when tissues are injured by viruses, bacteria, trauma, chemicals, heat, cold, or any other harmful stimuli. In such instances, the classic weapons of the immune system (T cells, B cells, macrophages) interface with cells and soluble products that are mediators of inflammatory responses (neutrophils, eosinophils, basophils, kinin and coagulation systems, and complement cascade). The ability of the immune system to discriminate between “self” and “non-self”(foreign) antigens is therefore vital to the functioning of the immune system as a specific defense against “non-self” antigens.

“Non-self” antigens are those antigens or substances entering a subject, or exist in a subject but are detectably different or foreign from the subject's own constituents, whereas “self” antigens are those which, in the healthy subject, are not detectably different or foreign from its own constituents. However, under certain conditions, including in certain disease states, an individual's immune system will identify its own constituents as “non-self,” and initiate an immune response against “self-antigens,” at times causing more damage or discomfort as from, for example, an invading microbe or foreign material, and often producing serious illness in a subject.

In another important embodiment, the inflammation is caused by an immune response against “self-antigen,” and the subject in need of treatment according to the invention has an autoimmune disease. “Autoimmune disease” as used herein, results when a subject's immune system attacks its own organs or tissues, producing a clinical condition associated with the destruction of that tissue, as exemplified by diseases such as uveitis, insulin-dependent diabetes mellitus, hemolytic anemias, rheumatic fever, Crohn's disease, Guillain-Barre syndrome, psoriasis, thyroiditis, Graves' disease, myasthenia gravis, autoimmune hepatitis, multiple sclerosis, systemic lupus erythematosus, etc. In some embodiments, the following are sepcifically excluded from the term autoimmune disease: arthritis (e.g., rheumatoid arthritis, collagen-induced arthritis), glomerulo-nephritis, lupus (e.g., system lupus erythematosus (SLE)), membranous nephropathy, and/or myasthenia gravis.

Autoimmune disease may be caused by a genetic predisposition alone, by certain exogenous agents (e.g., viruses, bacteria, chemical agents, etc.), or both. When the tissues in these areas become exposed to lymphocytes, their surface proteins can act as antigens and trigger the production of antibodies and cellular immune responses which then begin to destroy those tissues. Other autoimmune diseases develop after exposure of a subject to antigens which are antigenically similar to, that is cross-reactive with, the subject's own tissue. In rheumatic fever, for example, an antigen of the streptococcal bacterium, which causes rheumatic fever, is cross-reactive with parts of the human heart. The antibodies cannot differentiate between the bacterial antigens and the heart muscle antigens, consequently cells with either of those antigens can be destroyed.

In further embodiments, the inflammation is caused by an immune response against “non-self-antigens” (including antigens of necrotic self-material), and the subject in need of treatment according to the invention is a transplant recipient, has atherosclerosis, has suffered a an ischemic stroke and/or has an abscess and/or myocarditis. This is because after cell (or organ) transplantation or ischemic stroke, certain antigens from the transplanted cells (organs), or necrotic cells from the brain, can stimulate the production of immune lymphocytes and/or autoantibodies, which later participate in inflammation/rejection (in the case of a transplant), or attack brain target cells causing inflammation and aggravating the condition (Johnson et al., Sem. Nuc. Med. 1989, 19:238; Leinonen et al., Microbiol. Path.,1990, 9:67; Montalban et al., Stroke, 1991, 22:750).

Atherosclerosis can lead to ischemia-reperfusion (I/R) injury. Ischemia refers to a lack of oxygen due to inadequate perfusion of blood. One of the underlying mechanisms for I/R-induced injury is the hypoxic and reoxygenated environments created in affected tissues. Fluctuations in oxygen content as observed in these instances can create oxygen free radicals which have been reported to, among other things, modulate endothelial cell surface profile. Methods of the invention are, therefore, useful for treating cellular injury arising from ischemia/reperfusion, in some embodiments, associated with atherosclerosis. The methods also include treatment of injury that occurs in organs such as the brain, lungs, and kidneys as well as skeletal muscle. Injury to the vascular system can lead to a number of undesirable health conditions, including, for example, forms of atherosclerosis and arteriosclerosis that are associated with unwanted vascular smooth muscle cell proliferation.

The compositions of C1q inhibitors, provided herein, may be administered in combination with other therapeutic treatments.

The C1q inhibitors can be used alone as a primary therapy or in combination with other therapeutics as an adjuvant therapy to enhance the therapeutic benefits of other medical treatments. These other medical treatments include those used to treat inflammation and related tissue injury and autoimmune conditions as described above. These other medical treatments include agents such as anti-inflammatory agents, immunomodulators (e.g., immunosuppressants) as well as agents used to treat autoimmune disease, allergic reactions, ischemia, atherosclerosis, arteriosclerosis or other arterial conditions.

C1q inhibitors, therefore, can be administered to a subject along with an anti-inflammatory agent. Anti-inflammatory agents include: Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; Orpanoxin; Oxaprozin; s Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Zomepirac Sodium.

C1q inhibitors can also be administered along with immunosuppressants. Such immunosuppressants include: Azathioprine; Azathioprine Sodium; Cyclosporine; Daltroban; Gusperimus Trihydrochloride; Sirolimus; Tacrolimus.

C1q inhibitors can also be administered along with medication for treating allergic reactions. Such antiallergic medications include, antihistamines, corticosteroids and epinephrine.

C1q inhibitors can also be administered with agents for treating cerebral ischemia. Examples of therapeutics useful for treatment of cerebral ischemia include anticoagulation agents, antiplatelet agents, and thrombolytic agents.

Anticoagulants include, but are not limited to, heparin, warfarin, coumadin, dicumarol, phenprocoumon, acenocoumarol, ethyl biscoumacetate, and indandione derivatives.

Antiplatelet agents include, but are not limited to, aspirin, thienopyridine derivatives such as ticlopodine and clopidogrel, dipyridamole and sulfinpyrazone, as well as RGD mimetics and also antithrombin agents such as, but not limited to, hirudin.

Thrombolytic agents include, but are not limited to, plasminogen, a₂-antiplasmin, streptokinase, antistreplase, tissue plasminogen activator (tPA), and urokinase.

Other medical treatments can also include treatments used to inhibit complement activation. The C1q inhibitors can be used in conjunction with other complement inhibitors of any of the complement pathways. In certain embodiments, the other complement inhibitors are inhibitors of the classical pathway. In other embodiments, the other complement inhibitors inhibit the activation of the lectin (e.g., MBL or ficolins) or alternative complement pathways. For instance, the lectin or alternative complement pathway may initiate complement activation, lead to cellular injury and perhaps release novel epitopes to natural antibodies, which then would lead to activation of the classical pathway. The C1q inhibitors, provided herein, could therefore be used as an adjunctive therapy to other complement inhibition. Therefore, in some embodiments, the C1q inhibitors can be used with any agent that inhibits the activation of the lectin pathway or the alternative complement pathway (e.g., lectin pathway inhibitors or alternative complement pathway inhibitors).

C1q inhibitors can also be used in the treatment of cancer. The invention is based, in part, on the finding that inhibiting the classical complement pathway with C1q inhibitors would allow the MBL pathway to remove these apoptotic cells in an inflammatory/destructive cell removal process. Use of the C1q inhibitors, therefore, would lead to an exaggerated anti-tumor response, resulting in the killing of the apoptotic cells and neighboring cancer cells.

The cancer may be a malignant or non-malignant cancer. Cancers or tumors include but are not limited to biliary tract cancer; brain cancer; breast cancer; cervical cancer; choriocarcinoma; colon cancer; endometrial cancer; esophageal cancer; gastric cancer; intraepithelial neoplasms; lymphomas; liver cancer; lung cancer (e.g. small cell and non small cell); melanoma; neuroblastomas; oral cancer; ovarian cancer; pancreas cancer; prostate cancer; rectal cancer; sarcomas; skin cancer; testicular cancer; thyroid cancer; and renal cancer, as well as other carcinomas and sarcomas. In one embodiment the cancer is hairy cell leukemia, chronic myelogenous leukemia, cutaneous T-cell leukemia, multiple myeloma, follicular lymphoma, malignant melanoma, squamous cell carcinoma, renal cell carcinoma, prostate carcinoma, bladder cell carcinoma, or colon carcinoma.

Persons with or at risk of cancer can be treated with the C1q inhibitors provided herein. As used herein, the terms “treat” or “treating” with respect to cancer mean to eliminate cancer cells, inhibit their growth or otherwise reduce or eliminate symptoms as a result of the presence of the cancer. These terms also include the prevention of cancer in those subjects that are at risk of cancer but would not yet be diagnosed with cancer. C1q inhibitors can be administered to a person with or at risk of a cancer in an effective amount to treat the cancer. C1q inhibitors can be provided alone or in combination with an anti-cancer agent. Anti-cancer agents include, but are not limited to, chemotherapeutic agents, radiotherapeutics and protein toxins. Preferably, the anti-cancer agents are those which promote apoptosis of the cancer cells. Anti-cancer agents can be administered to a subject in need thereof prior to the administration of the C1q inhibitor. In other instances, the anti-tumor agent is administered concomitantly with the C1q inhibitor. In other instances the anti-tumor agent is administered subsequent to the C1q inhibitor.

An “apoptotic chemotherapeutic agent” as used herein is a group of molecules which function by a variety of mechanisms to induce apoptosis in rapidly dividing cells. Apoptotic chemotherapeutic agents are a class of chemotherapeutic agents which are well known to those of skill in the art. Chemotherapeutic agents include those agents disclosed in Chapter 52, Antineoplastic Agents (Paul Calabresi and Bruce A. Chabner), and the introduction thereto, 1202-1263, of Goodman and Gilman's “The Pharmacological Basis of Therapeutics”, Eighth Edition, 1990, McGraw-Hill, Inc (Health Professions Division), incorporated herein by reference. Suitable chemotherapeutic agents may have various mechanisms of action. The classes of suitable chemotherapeutic agents include (a) Alkylating Agents such as nitrogen mustard (e.g. mechlorethamine, cylophosphamide, ifosfamide, melphalan, chlorambucil), ethylenimines and methylmelamines (e.g. hexamethylmelamine, thiotepa), alkyl sulfonates (e.g. busulfan), nitrosoureas (e.g. carmustine which is also known as BCNU, lomustine which is also known as CCNU semustine which is also known as methyl-CCNU, chlorozoticin, streptozocin), and triazines (e.g. dicarbazine which is also known as DTIC); (b) Antimetabolites such as folic acid analogs (e.g. methotrexate), pyrimidine analogs (e.g. 5-fluorouracil floxuridine, cytarabine, and azauridine and its prodrug form azaribine), and purine analogs and related materials (e.g. 6-mercaptopurine, 6-thioguanine, pentostatin); (c) Natural Products such as the vinca alkaloids (e.g. vinblastine, Vincristine), epipodophylotoxins (e.g. etoposide, teniposide), antibiotics (.e.g dactinomycin which is also known as actinomycin-D, daunorubicin, doxorubicin, bleomycin, plicamycin, mitomycin, epirubicin, which is 4-epidoxorubicin, idarubicin which is 4-dimethoxydaunorubicin, and mitoxanthrone), enzymes (.e.g L-asparaginase), and biological response modifiers (e.g. Interferon alfa); (d) Miscellaneous Agents such as the platinum coordination complexes (e.g. cisplatin, carboplatin), substituted ureas (e.g. hydroxyurea), methylhydiazine derivatives (e.g. procarbazine), adreocortical suppressants (e.g. mitotane, aminoglutethimide) taxol; (e) Hormones and Antagonists such as adrenocorticosteroids (e.g. prednisone or the like), progestins (e.g. hydroxyprogesterone caproate, medroxyprogesterone acetate, megestrol acetate), estrogens (e.g. diethyestilbestrol, ethinyl estradiol, and the like), antiestrogens (e.g. tamoxifen), androgens (e.g. testosterone propionate, fluoxymesterone, and the like), antiandrogens (e.g. flutamide), and gonadotropin-releasing hormone analogs (e.g. leuprolide) and (F) DNA damaging compounds such as adriamycin.

Other specific examples of anti-cancer agents include: Acivicin; Aclarubicin; Acodazole Hydrochloride; Acronine; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Podofilox; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Taxotere; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride.

The method for inhibiting classical complement activation may also be used for a variety of in vitro and in vivo purposes. The method may be used, for instance, as an in vitro screening assay. The in vitro screening assay may be used to identify compounds which function as a C1q inhibitor or to detect susceptibility of a subject to treatment with a C1q inhibitor. In order to screen a subject for susceptibility to treatment with a C1q inhibitor, serum is isolated from the subject and the presence of C1q is detected. If C1q is present, then the person is susceptible to classical complement activation. If this is the case, then the subject is susceptible to treatment with an C1q inhibitor. Once the subjects are identified which are susceptible to treatment with an C1q inhibitor, the subjects can then be treated according to the methods of the invention.

The step of “contacting” as used herein refers to the addition of the C1q inhibitor to a sample containing C1q. The sample may be any biological specimen. The step of contacting refers to the addition of the C1q inhibitor in such a manner that it will be able to bind C1q if present in the sample.

According to the methods of the invention, the compositions may be administered in a pharmaceutically acceptable composition. In general, pharmaceutically-acceptable carriers for monoclonal antibodies, antibody fragments, and peptides are well-known to those of ordinary skill in the art. As used herein, a pharmaceutically-acceptable carrier means a non-toxic material that does not interfere with the effectiveness of the biological activity of the active ingredients, i.e., the ability of the C1q inhibitor to inhibit classical complement activation. Pharmaceutically acceptable carriers include diluents, fillers, salts, buffers, stabilizers, solubilizers and other materials which are well-known in the art. Exemplary pharmaceutically acceptable carriers for peptides in particular are described in U.S. Pat. No. 5,211,657. The peptides of the invention may be formulated into preparations in solid, semi-solid, liquid or gaseous forms such as tablets, capsules, powders, granules, ointments, solutions, depositories, inhalants (e.g., aerosols) and injections, and usual ways for oral, parenteral or surgical administration. The invention also embraces locally administering the compositions of the invention, including as implants. Administration can also be systemic. In certain embodiments, the C1q inhibitors provided can be conjugated to a targeting molecule for delivery to a specific site in the subject. Targeting molecule include, but are not limited to, molecules such as a ligand for a receptor on the target cell. Targeting molecules, therefore include molecules (e.g., antibodies) specific for tumor antigens.

According to the methods of the invention the compositions can be administered by injection by gradual infusion over time or by any other medically acceptable mode. The administration may, for example, be intravenous, intraperitoneal, intramuscular, intracavity, subcutaneous or transdermal. Preparations for parenteral administration includes sterile aqueous or nonaqueous solutions, suspensions and emulsions. Examples of nonaqueous solvents are propylene glycol, polyethylene glycol, vegetable oil such as olive oil, an injectable organic esters such as ethyloliate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers, (such as those based on Ringer's dextrose), and the like. Preservatives and other additives may also be present such as, for example, antimicrobials, antioxidants, chelating agents, and inert gases and the like. Those of skill in the art can readily determine the various parameters for preparing these alternative pharmaceutical compositions without resorting to undue experimentation. When the compositions of the invention are administered for the treatment of pulmonary disorders the compositions may be delivered for example by aerosol.

The compositions of the invention are administered in therapeutically effective amounts. As used herein, an “effective amount” of the inhibitor of the invention is a dosage which is sufficient to inhibit the increase in, maintain or even reduce the amount of undesirable classical complement activation. The effective amount, in some aspects of the invention, is sufficient to produce the desired effect of killing cancer cells, reducing tumor size or limiting the growth of a tumor. An effective amount can also produce the desired effect of inhibiting associated cellular injury until the symptoms associated with the classical complement mediated disorder are ameliorated or decreased. Preferably an effective amount of the peptide is an effective amount for preventing cellular injury. Generally, a therapeutically effective amount may vary with the subject's age, condition, and sex, as well as the extent of the disease in the subject and can be determined by one of skill in the art. The dosage may be adjusted by the individual physician or veterinarian in the event of any complication. A therapeutically effective amount typically will vary from about 0.01 mg/kg to about 500 mg/kg, were typically from about 0.1 mg/kg to about 200 mg/kg, and often from about 0.2 mg/kg to about 20 mg/kg, in one or more dose administrations daily, for one or several days (depending of course of the mode of administration and the factors discussed above). A preferred concentration of the inhibitor is a concentration which is equimolar to the concentration of C1q in the plasma of a subject. The normal plasma concentration of C1q can be assessed clinically. A normal range of C1q is 50-100 μg/ml C1q/plasma.

One of skill in the art can determine what an effective amount of a C1q inhibitor is by screening the ability of the inhibitor to inhibit the classical complement activation in an in vitro assay. The activity of the inhibitor can be defined in terms of the ability of the inhibitor to inhibit classical complement activation. An exemplary assay for measuring the ability of a putative C1q inhibitor of the invention to inhibit classical complement activation has been discussed above. The exemplary assay is predictive of the ability of an inhibitor to inhibit classical complement activation in vivo and, hence, can be used to select inhibitors for therapeutic applications.

The C1q inhibitors may be administered in a physiologically acceptable carrier. The term “physiologically-acceptable” refers to a non-toxic material that is compatible with the biological systems such of a tissue or organism. The physiologically acceptable carrier must be sterile for in vivo administration. The characteristics of the carrier will depend on the route of administration.

The C1q binding peptides linked to a therapeutic moiety are also provided. Therapeutic moieties include drugs such as antitumor agents. Antitumor agents, preferably are agents which promote apoptotic activity of cancer cells. These agents can include cytotoxic agents such as chemotherapeutic agents and radiotherapies. Therapeutic moieties also include immunomodulating agents (e.g., immunosuppressants) and anti-inflammatory agents provided above.

The invention further provides detectably labeled, immobilized and toxin conjugated forms of the C1q binding peptides, antibodies and fragments thereof. The antibodies may be labeled using, for example, radiolabels, fluorescent labels, enzyme labels, free radical labels, avidin-biotin labels, or bacteriophage labels, using techniques known to the art (Chard, Laboratory Techniques in Biology, “An Introduction to Radioimmunoassay and Related Techniques,” North Holland Publishing Company (1978).

Typical fluorescent labels include fluorescein isothiocyanate, rhodamine, phycoerythrin, phycocyanin, allophycocyanin, and fluorescamine.

Typical chemiluminescent compounds include luminol, isoluminol, aromatic acridinium esters, imidazoles, and the oxalate esters.

Typical bioluminescent compounds include luciferin, and luciferase. Typical enzymes include alkaline phosphatase, β-galactosidase, glucose-6-phosphate dehydrogenase, maleate dehydrogenase, glucose oxidase, and peroxidase.

The coupling of one or more molecules to the C1q binding peptides is envisioned to include many chemical mechanisms, for instance covalent binding, affinity binding, intercalation, coordinate binding, and complexation. The therapeutic moieties can be attached to the C1q binding peptides by standard protocols known in the art.

The covalent binding can be achieved either by direct condensation of existing side chains or by the incorporation of external bridging molecules. Many bivalent or polyvalent agents are useful in coupling protein molecules to other proteins, peptides or amine functions, etc. For example, the literature is replete with coupling agents such as carbodiimides, diisocyanates, glutaraldehyde, diazobenzenes, and hexamethylene diamines. This list is not intended to be exhaustive of the various coupling agents known in the art but, rather, is exemplary of the more common coupling agents.

Radionuclides typically are coupled to an antibody by chelation. For example, in the case of metallic radionuclides, a bifunctional chelator is commonly used to link the isotope to the antibody or other protein of interest. Typically, the chelator is first attached to the antibody, and the chelator-antibody conjugate is contacted with the metallic radioisotope.

Further, a method of detecting C1q in a biological sample (e.g., serum) is also provided. This method involves providing a C1q binding peptide that binds to a C1q. The C1q binding peptide is bound to a label that permits the detection of C1q in the sample upon binding of the C1q binding peptide to C1q. The biological sample is contacted with the labeled C1q binding peptide under conditions effective to permit binding of the C1q binding peptide to C1q in the biological sample. The presence of the C1q-C1q binding peptide complex is then detected by detection of the label. Many labels which can be used to observe the C1q binding peptide interacting with C1q are known in the art.

The following examples are provided to illustrate specific instances of the practice of the present invention and are not to be construed as limiting the present invention to these examples. As will be apparent to one of ordinary skill in the art, the present invention will find application in a variety of compositions and methods.

### EXAMPLES

C1q Antibody Generation

Two male C1q knockout mice (David Pinsky (Columbia University, NY) via Marina Botto (England)) were immunized intraperitoneally with approximately 50 μg human C1q (Advanced Research Technologies, Cambridge, Mass.) emulsified with Titermax adjuvant. At two-week intervals the mice were boosted with 25 μg of human C1q in PBS for a total of two booster injections. Antibody titers were checked 10 days after second booster injection. Four days prior to removal of the spleen and fusion, mice were again boosted with 20 μg human C1q in PBS. Fusion was performed in presence of PEG with P301 mouse myelinoma cells. Fused cells were plated in 96 well plates at 2.5×10⁶ cells/ml. The cells were fed daily for 3 days then every 2-3 days. Antibody capture ELISAs were done 10 days after fusion to identify anti-C1q positive wells. Isotyping was done on all positive wells to remove IgM class wells. Functional CH50 assays were done 12 days after fusion. Monoclonal status was achieved by limited dilution techniques.

Anti-Human C1q Hemolytic Complement Assay (CH₅₀)

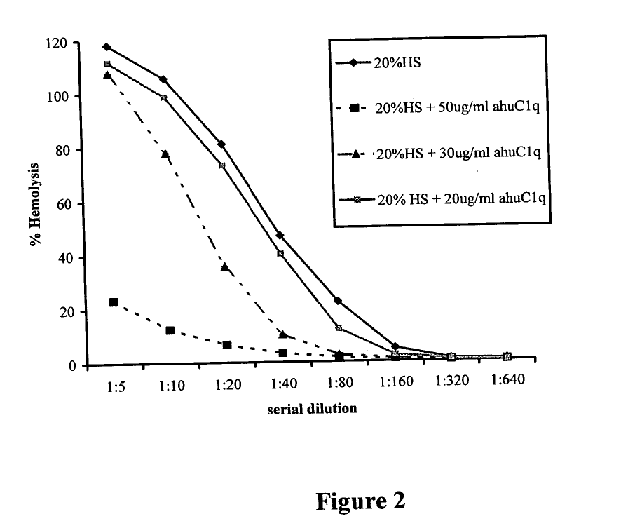

Human serum from healthy donors was stored at −80° C. until use. Serum (20%), or serum aliquots containing varying concentrations (20, 30, and 50 μg/ml respectively) of anti-human C1q P1 H10 mAb were incubated at 25° C. for 45 minutes. In each row of a 96-well round bottom microtiter plates, serum aliquots was serially diluted in gelatin-veronal buffered saline (GVB⁺⁺) to give a series of dilutions (x-axis), at a final volume of 100 μl. With the plate on ice to retard activation, 30 μl aliquots of antibody-sensitized chicken erythrocytes (Colorado Serum Company, Denver, Colo.) in GVB⁺⁺ at 1×10⁸ cells/ml were added to each well and mixed. A negative control (cells+GVB⁺⁺) and positive control (cells+1% TritonX) were included in each plate. The plates were incubated at 37° C. for 30 minutes, and centrifuged for 10 minutes. Supernatants (75 μl) were transferred to flat bottom plates and read at 405 nm on a SpectraMax plus microtiter plate reader (Molecular Devices, Sunnyvale, Calif.). The percent hemolysis was calculated for each serum dilution: (average sample−average negative control)/(average positive control−average negative control)×100. Each plate was run in triplicate, +/−SE, n=3. FIG. 2 illustrates that hemolysis decreased with increasing antibody concentration.

MBL-Dependent C3 Deposition Enzyme-Linked Immunosorbent Assay (ELISA)

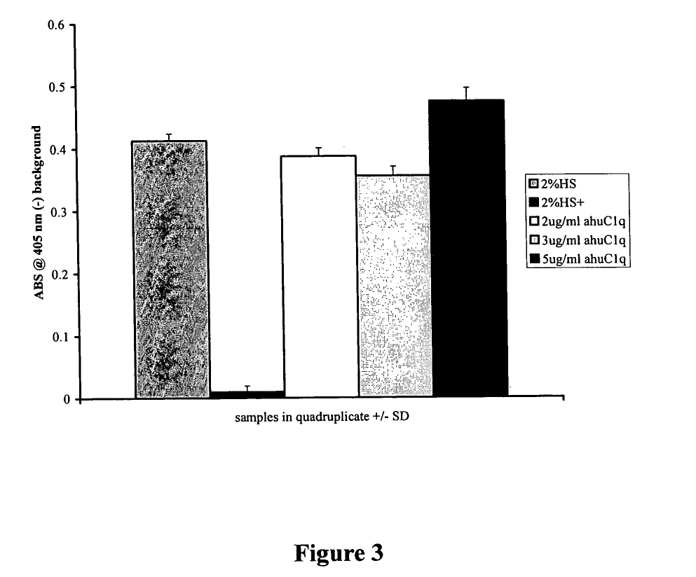

Radioimmunoassay 96-well plates were coated with 100 μl of 0.5 mg/ml mannan in sodium carbonate/bicarbonate buffer pH 9.6 and incubated overnight at 4° C. Plates were then washed three times with PBS buffer containing 0.5% Tween-20 and washed once with PBS buffer followed by a second wash with veronal-buffered saline (VBS: 141 mmol/l NaCl and 1.8 mmol/l sodium barbital). Human serum (2%), or 2% human serum containing various concentrations of anti-human C1q (2, 3, 4, and 10 μg/ml respectively) was added to each well and incubated for 30 minutes at 37° C. Plates were then washed four times with PBS buffer containing 0.5% Tween-20. Human C3 deposition was detected by addition of 50 μl of 1:2000 dilution of horseradish peroxidase-conjugated goat anti-human C3 (ICN, Costa Mesa, Calif.) to each well and incubated at 25° C. for 1 hour. The plates were washed four times and 100 μl of 2,2′-azino-bis(3-ethylbenzthiazoline-6-sulphonic) acid (ABTS) was added to each well and the plates were read at 405 nm using a SpectraMax plus microplate reader (Molecular Devices, Sunnyvale Calif.). GlcNAc (50 mmol/l), an inhibitor of the lectin complement pathway, was added to some wells to inhibit lectin pathway activation (2% HS⁺). Non-specific background absorbance consisted of wells containing VBS buffer without sera. All ELISA experiments were performed in quadruplicate, +/−SE, n=3. FIG. 3 illustrates that C3 deposition was inhibited most significantly with the use of an inhibitor of the lectin complement pathway, GlcNAc. This indicates the P1 H10 mAb is specific for the classical complement pathway.

IP of Human C1q from Sera Using Anti-Human C1q (P1 H10 mAb)

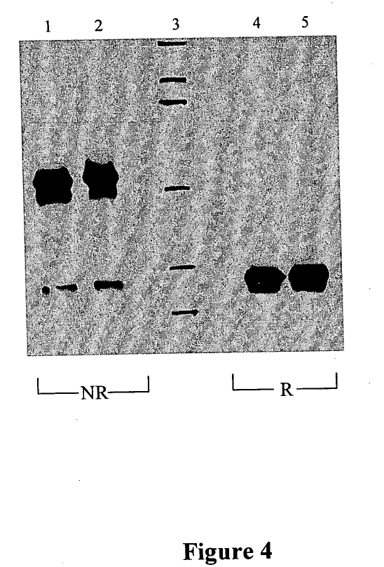

P1 H10 anti-human C1q mAb was coupled to activated sepharose (Pharmacia) at a final concentration of 3 mg/ml of P1 H10 on the beads. Human C1q was then immunoprecipitated from sera using P1 H10 beads overnight at 4° C. Beads were then centrifugated, washed, and resuspended in either reducing (R—lanes 4 and 5) or non-reducing (NR—lanes 1 and 2) sample buffer, boiled and loaded onto SDS-Page gels. Gels were transferred to nitrocellulose and western blot was performed using a polyclonal anti-human C1q-HRP antibody (1:1000). Blots were developed using SuperSignal ECL kit (Pierce, Rockford, Ill.). Lane 1—IP of human C1q (Advanced Research Technologies), NR. Lane 2—IP of human C1q isolated from human sera, NR. Lane 3—BioRad Broad Range SDS-Page pre-stained molecular weight marker. Lane 4—IP of human C1q (Advanced Research Technologies), R. Lane 5—IP of human C1q isolated from human sera, R. The results are provided in FIG. 4.

Representative Sensogram of the Binding of Anti-Human C1q P1 H10 mAb to Immobilized Human C1q

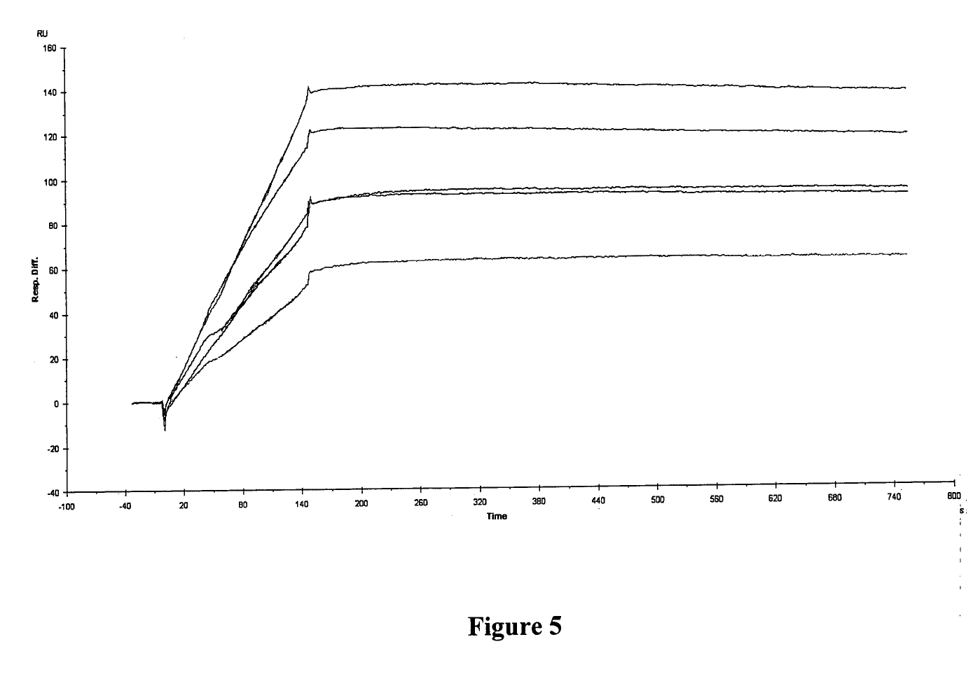

Five different concentrations of mAb anti-human C1q P1 H10 were injected over immobilized human C1q (Advanced Research Technologies) on a BIACORE apparatus for 120 seconds, and allowed to dissociate for 500 seconds, respectively. Each sensogram represents the relative changes in response units (RU) after subtraction of the control flow cell upon the changes of time. The curves were then x-transformed to the start of injection and y-transformed to the average baseline prior to the start of injection. Kinetics of anti- huC1q with huC1q was calculated using BIAEvaluation software, n=3. Table 1 below provides the kinetic constants from the analysis of the binding of P1 H10 mAb to human C1q. The plot is provided in FIG. 5.

TABLE 1
Kinetic Constants for P1 H10 mAb Binding to hC1q

| | | | | RI | Conc. of | | | | | Chi2 |
|---|---|---|---|---|---|---|---|---|---|---|
| | ka(l/Ms) | kd(l/s) | Rmax (RU) | (RU) | analyte | KA (l/M) | KD (M) | Req (RU) | kobs (l/s) | 0.236 |
| kinetics ahuClq − | 1.14E+05 | 8.02E−05 | 2.66E+02 | 50.1 | 2.67E−08 | 1.42E+09 | 7.02E−10 | 2.59E+02 | 3.13E−03 | |
| mw Fc = 4-3 − 1 | | | | | | | | | | |
| kinetics ahuClq − | 4.28E+03 | 8.48E−05 | 1.69E+04 | 47.3 | 1.33E−08 | 5.05E+07 | 1.98E−08 | 6.77E+03 | 1.42E−04 | |
| mw Fc = 4-3 − 2 | | | | | | | | | | |
| kinetics ahuClq − | 5.35E+03 | 3.16E−05 | 3.22E+03 | 20 | 2.00E−08 | 1.69E+08 | 5.90E−09 | 2.49E+03 | 1.39E−04 | |
| mw Fc = 4-3 − 3 | | | | | | | | | | |
| kinetics ahuClq − | 8.66E+05 | 5.23E−05 | 113 | 29 | 6.67E−09 | 1.66E+10 | 6.04E−11 | 112 | 5.83E−03 | |
| mw Fc = 4-3 − 5 | | | | | | | | | | |
| kinetics ahuClq − | 2.62E+05 | 1.43E−05 | 683 | 31.8 | 3.33E−09 | 1.83E+10 | 5.46E−11 | 672 | 8.86E−04 | |
| mw Fc = 4-3 − 6 | | | | | | | | | | |

Avg KD: 3-9 nM

Myocardial Ischemia-Reperfusion Injury is Dependent on Lectin Complement Activation

Complement C5 activation mediates myocardial injury following myocardial ischemia-reperfusion (MI/R). Complement activation is thought to result from natural antibodies binding to neo-epitopes on injured cells, resulting in classical pathway activation and PMN infiltration. Therefore, the contribution of the lectin and classical pathway in MI/R injury was evaluated using mannose binding ligand (MBL)-null (MBL-A/CKO), C1q-deficient (C1qKO), C2- and factor B-deficient (C2/Bf) or wild-type (WT) mice.

The LAD of each experimental animal was reversibly ligated for 30 min, followed by 3 h of reperfusion. After 3 h the LAD was re-ligated to establish healthy tissue and area at risk myocardium. Hearts were then stained for infarction and C3 or C1q deposition. Myocardial infarction (MI) is expressed as a percentage of the area at risk (infarct/area at risk×100). MI/R induced significantly larger MI in WT, compared to C2/Bf mice (33±2 vs. 12±2%, respectively; p<0.05). Addition of human C2 to C2/Bf significantly increased MI to 43×9%, comparable to WT mice and demonstrating that MI/R injury is mediated via classical and/or lectin pathway activation. WT or C1qKO mice undergoing MI/R demonstrated myocardial C3 deposition, thus demonstrating complement activation. WT mice following MI/R demonstrated C1q deposition in the myocardium, whereas C1qKO mice did not. C1qKO mice demonstrated a MI of 47±7%, and anti-C5 mAb treatment significantly (p<0.05) reduced the infarction to 12±4%. Additionally, MBL null mice demonstrate significantly decreased MI (4+2%; p<0.05) in comparison to WT mice following experimental MI/R. However, addition of recombinant MBL to MBL null mice significantly increased MI to 25±8%.

Collectively, these data demonstrate that complement activation contributes to MI/R injury. The complement system is not activated during MI/R by a C1q dependent mechanism, yet C1q is deposited. Importantly, MBL null mice demonstrate little to no infarct following experimental MI/R, yet injury is re-established upon addition of MBL. Ultimately, these findings demonstrate the therapeutic potential of lectin complement pathway blockade in MI/R and ischemic heart disease.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

All references, patents and patent publications that are recited in this application are incorporated in their entirety herein by reference.

1. A hybridoma cell line deposited under ATCC Accession No. PTA-5226.

2. An antibody produced by the hybridoma cell line of claim 1.

3. An isolated C1q binding peptide, which is a monoclonal antibody produced by the hybridoma cell line of claim 1 or an antigen-binding fragment thereof.

4. The isolated C1q binding peptide of claim 3, wherein the antigen-binding fragment is selected from the group consisting of a F(ab′)₂ fragment, a Fd fragment, a Fab fragment, and a Fv fragment.

5. The isolated C1q binding peptide of claim 3, wherein the antigen-binding fragment is a CDR.

6. The isolated C1q binding peptide according to claim 5, wherein the CDR is CDR3.

7. The isolated C1q binding peptide according to claim 6, wherein the CDR3 is the heavy chain CDR3.

8. The isolated C1q binding peptide according to claim 3, wherein the antigen-binding fragment is a light chain CDR2 region.

9. The isolated C1q binding peptide according to claim 3, wherein the antigen-binding fragment is a light chain CDR1 region.

10. The isolated C1q binding peptide according to claim 3, wherein the antigen-binding fragment is a heavy chain CDR2 region.

11. The isolated C1q binding peptide according to claim 3, wherein the antigen-binding fragment is a heavy chain CDR1 region.

12. The isolated C1q binding peptide according to claim 3, wherein the monoclonal antibody is bound to at least one therapeutic moiety.

13. The isolated C1q binding peptide according to claim 12, wherein the therapeutic moiety is a drug.

14. An isolated C1q binding peptide which selectively binds to a human C1q epitope defined by a monoclonal antibody produced by the hybridoma cell line deposited under ATCC Accession No: PTA-5226.

15. A composition, comprising:
the isolated C1q binding peptide according to claim 3, and a pharmaceutically acceptable carrier.

16. The composition according to claim 15, wherein the isolated C1q binding peptide is a C1q inhibitor that inhibits classical complement activation.

17. The composition according to claim 15, wherein the isolated C1q binding peptide is an intact soluble monoclonal antibody.

18. The composition according to claim 16, further comprising a drug for the treatment of the classical complement mediated disorder.

19. The composition according to claim 18, wherein the classical complement mediated disorder is an inflammatory condition, and the drug is an anti-inflammatory agent or immunomodulator.

20. The composition according to claim 18, wherein the classical complement mediated disorder is cancer, and the drug is an anti-cancer agent that induces apoptosis.

21. A method for inhibiting classical complement activation, comprising contacting a C1q molecule with an effective amount of the composition according claim 15 to inhibit classical complement activation.

22. The method of claim 21, wherein the composition is administered to a subject in an amount effective to inhibit classical complement activation.

23. The method of claim 22, wherein the subject is a mammal.

24. The method of claim 23, wherein the mammal is an human.

25. A method of treating an inflammatory condition, comprising:
administering to a subject with an inflammatory condition or at risk thereof the composition according to claim 15 in an amount effective to treat or prevent the inflammatory condition.

26. The method of claim 25, wherein the inflammatory condition is inflammation that results from the administration of a therapeutic agent.

27. The method of claim 25, wherein the inflammatory condition is inflammation that results from the administration of a liposome or lipid formulation.

28. The method of claim 25, wherein the inflammation results in an allergic reaction.

29. A method of treating an autoimmune disorder, comprising:
administering to a subject with an autoimmune disorder or at risk thereof the composition according to claim 15 in an amount effective to treat or prevent the autoimmune disorder.

30. A method of treating an autoimmune disorder, wherein the autoimmune disorder is not arthritis, glomerulo-nephritis, lupus, membranous nephropathy or myasthenia gravis, comprising:
administering to a subject with an autoimmune disorder or at risk thereof a C1q inhibitor in an amount effective to treat or prevent the autoimmune disorder.

31. A method of treating cancer, comprising:
administering to a subject with cancer or at risk thereof a C1q inhibitor and an anti-cancer agent that induces apoptosis in an amount effective to treat cancer.

32. A method of treating cancer, comprising:
administering to a subject with cancer or at risk thereof a C1q inhibitor and an anti-cancer agent that induces apoptosis in an amount effective to treat cancer, wherein the C1q inhibitor is the isolated C1q binding peptide of the compositions according to claim 15.

33. The method of claim 31, wherein the C1q inhibitor and the anti-cancer agent are administered concomitantly.

34. The method of claim 31, wherein the cancer is selected from the group consisting of bladder cancer, pancreatic cancer, lung cancer, kidney cancer, sarcoma, breast cancer, brain cancer, colon cancer, liver cancer, stomach cancer, ovarian cancer, prostate cancer, testicular cancer, and melanoma.

35. A method of treating ischemia reperfusion, comprising:
administering to a subject with ischemia reperfusion or at risk thereof a C1q inhibitor in an amount effective to treat ischemia reperfusion, wherein the ischemia reperfusion is not cardiac or gastrointestinal ischemia reperfusion.

36. The method of claim 35, wherein the ischemia reperfusion occurs in the brain, lungs, kidneys or skeletal muscle.

37. The method of claim 35, wherein the C1q inhibitor is the isolated C1q binding peptide of the compositions according to claim 15.

38. A method for detecting the presence of C1q in a sample comprising:
contacting the sample with an isolated C1q binding peptide according to claim 3, and
detecting the isolated C1q binding peptide-C1q complex, wherein the presence of a complex in the sample is indicative of the presence of C1q in the sample.
